(19) United States
(12) Patent Application Publication
(10) Pub. No.: US 2006/0009469 A1
(43) Pub. Date: Jan. 12, 2006

(54) Particulate-stabilized injectable pharmacutical compositions of posaconazole

(76) Inventors: Leonore Witchey-Lakshmanan, Piscataway, NJ (US); Sydney Ugwu, Gurnee, IL (US); Varda Sandweiss, Forest Hills, NY (US); Catherine Hardalo, Morristown, NJ (US); Roberta S. Hare, Gillette, NJ (US); Gopal Krishna, North Brunswick, NJ (US); Zaiqi Wang, Edison, NJ (US); Marco Taglietto, Watchung, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(21) Appl. No.: 11/140,294

(22) Filed: May 27, 2005

Related U.S. Application Data
(60) Provisional application No. 60/575,126, filed on May 28, 2004.

(51) Int. Cl. A61K 31/496

(52) U.S. Cl. 514/254.07

(57) ABSTRACT

The present invention provides formulations useful for treating infections, in particular, formulations that include the active pharmaceutical ingredient posaconazole in an injectable suspension that is stable when subjected to terminal steam sterilization.

This application claims priority benefits of application No. 60/575,126 filed May 28, 2004, the entire disclosure of which is incorporated herein by reference.

### FIELD OF THE INVENTION

The present invention relates to formulations useful for treating infections. Specifically, these formulations include the active pharmaceutical ingredient posaconazole in an injectable suspension that is stable when subjected to terminal steam sterilization, and throughout the shelf life of the product.

### BACKGROUND OF THE INVENTION

Posaconazole, an anti-fungal agent, represented by the following chemical structural formula 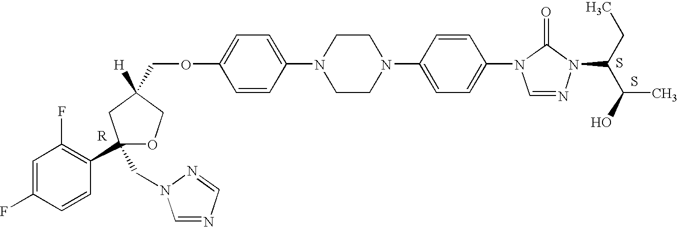
is being developed as an oral suspension (40 mg/ml) under the trademark NOXAFIL® by Schering Corporation, Kenilworth, N.J. See, for example, U.S. Pat. Nos. 5,703,079, 5,661,151, WO 02/80678 published Oct. 17, 2002, and EP 1 372 394 published Jan. 2, 2004. In addition, other formulations of posaconazole have been disclosed. A solid (capsule/tablet) of posaconazole is disclosed in U.S. Pat. Nos. 5,972,381 and 5,834,472. Lastly, a topical form of posaconazole, e.g., a lotion, cream, ointment, or “lacquer nail polish” is contemplated based on other similar formulations, e.g., U.S. Pat. No. 4,957,730 (PENLAC® available from Dermik®).

Certain aspects of stabilization of micronized particles in pharmaceutical compositions are addressed in the literature. For example, U.S. Pat. No. 5,858,410 discloses pharmaceutical compositions containing particles of active agents of average diameter less than 5 microns, having been comminuted, without prior conversion into a melt, by using a piston-gap homogenizer. U.S. patent application Ser. No. 10/440,368 discloses the use of a phospholipid surface active agent to stabilize microparticles of solid fenofibrate in an orally administered pharmaceutical composition. U.S. Pat. No. 5,091,188 discloses the use of phospholipids, to prevent coalescence of microcrystalline active agents in injectable pharmaceutical compositions. Examples of disclosed phospholipids include lecithin, phosphatidic acid, phosphatidyl ethanolamine, cholesterol, stearylamine, glycolipids and mono-glycerides.

None of the aforementioned references however, discloses an injectable suspension of posaconazole, that is stable when subjected to terminal steam sterilization and throughout the shelf life of the product. There is a need for such a formulation as it is desirable to ensure the physical stability of the sterilized end product.

### SUMMARY OF THE INVENTION

The present invention provides formulations of posaconazole that are stable when subjected to terminal steam sterilization. These formulations are useful for the treatment of infections. In particular, an aqueous injectable suspension of posaconazole that is homogenously suspended in vehicle with the aid of a phospholipid. In addition a thermoprotectant agent is employed to reduce autoclave-induced particle size growth, as well as a buffer system to stabilize the phospholipid during autoclaving. The formulations provided remain stable after 20 minutes of autoclaving at 121° C. and after subsequent storage at 4° C. to 40° C. for at least 6 months.

The present invention provides formulations comprising a suspension of posaconazole, stabilized by a phospholipid, in a mixture comprising a thermoprotectant, and a buffer system.

In some embodiments, the formulation has been sterilized by autoclaving or by irradiation.

In some embodiments, the buffer system comprises sodium phosphate, which may be provided as sodium phosphate monobasic monohydrate, sodium phosphate dibasic anhydrous, or the combination of the two.

In some embodiments, the buffer system comprises an organic buffer.

In some embodiments, the buffer system comprises at least one of histidine, citric acid, glycine, sodium citrate, ammonium sulfate, or acetic acid.

In some embodiments, the buffer system maintains a pH of about 3.0 to about 9.0.

In some embodiments, the buffer system maintains a pH of about 6.0 to about 8.0.

In some embodiments, the buffer system maintains a pH of about 6.4 to about 7.6.

In some embodiments, the phospholipid comprises a natural phospholipid.

In some embodiments, the phospholipid comprises a synthetic phospholipid.

In some embodiments, the phospholipid comprises a natural phospholipid and a synthetic phospholipid.

In some embodiments, the phospholipid comprises 1-palmitoyl-2-oleoyl-sn-glycero-3-phosphocholine (POPC).

In some embodiments, the thermoprotectant comprises trehalose.

In some embodiments, the phospholipid comprises 1-palmitoyl-2-oleoyl-sn-glycero-3-phosphocholine (POPC), the thermoprotectant comprises trehalose, and the buffer system comprises sodium phosphate monobasic monohydrate, sodium phosphate dibasic anhydrous, or the combination of sodium phosphate monobasic monohydrate and sodium phosphate dibasic anhydrous.

In some embodiments, the posaconazole has a particle size distribution whose median value is between about 1.0 and about 8.0 microns, with not more than about 3000 particles of 10 microns or greater size and not more than about 300 particles of 25 microns or greater size.

In some embodiments, the posaconazole has a particle size distribution whose median value is between about 1.0 and about 5.0 microns, with not more than about 3000 particles of 10 microns or greater size and not more than about 300 particles of 25 microns or greater size.

In some embodiments, the posaconazole has a particle size distribution whose median value is between about 1.2 and about 4.5 microns, with not more than about 3000 particles of 10 microns or greater size and not more than about 300 particles of 25 microns or greater size.

In some embodiments, the formulation has ingredients comprising:

| Ingredient | Concentration range | |
|---|---|---|
| Posaconazole | about 50 | mg/ml |
| POPC | about 40 | mg/ml |
| Sodium Phosphate, | 0.345 | mg/ml |
| monobasic, monohydrate, | | |
| USP | | |
| Sodium Phosphate, dibasic, | 1.065 | |
| anhydrous, USP | | |
| Trehalose | 250 | mg/ml |
| Water for Injection, USP q.s. | 1 | ml |
| ad | | |

In some embodiments, the formulation has ingredients comprising:

| Ingredient | Concentration range | |
|---|---|---|
| Posaconazole | about 1 to about 100 | mg/ml |
| POPC | about 10 to about 60 | mg/ml |
| Sodium Phosphate, | about 0.01 to about 0.6 | mg/ml |
| monobasic, monohydrate, | | |
| USP | | |
| Sodium Phosphate, dibasic, | about 0.04 to about 1.5 | mg/ml |
| anhydrous, USP | | |
| Trehalose | about 10 to about 300 | mg/ml |
| Water for Injection, USP q.s. | about 1 | ml |
| ad | | |

In some embodiments, the formulation has ingredients comprising:

| Ingredient | Concentration range | |
|---|---|---|
| Posaconazole | about 40 to about 60 | mg/ml |
| POPC | about 20 to about 50 | mg/ml |
| Trehalose | about 100 to about 250 | mg/ml |
| Water for Injection, USP q.s. | about 1 | ml |
| ad | | |

In some embodiments, the formulation has ingredients comprising:

| Ingredient | Concentration | |
|---|---|---|
| Posaconazole | 50 | mg/ml |
| POPC | 40 | mg/ml |
| Histidine | 3 | mg/ml |
| Citric acid monohydrate | 0.24 | mg/ml |
| Trehalose | 250 | mg/ml |
| Water q.s. ad | 1 | ml |

at a pH of about 6.4.

In some embodiments, the formulation has ingredients further comprising an antioxidant.

In some embodiments, the antioxidant comprises propyl gallate at a concentration of about 0.02 to about 0.005 mg/ml.

In some embodiments, the antioxidant comprises butylated hydroxytoluene at a concentration of about 0.1 to about 0.02 mg/ml.

In some embodiments, the antioxidant comprises alpha-D-tocopherol at a concentration of about 0.5 to about 0.01 mg/ml.

In some embodiments, the formulation has ingredients comprising:

| Ingredient | Concentration | |
|---|---|---|
| Posaconazole | 50 | mg/ml |
| POPC | 40 | mg/ml |
| Histidine | 3 | mg/ml |
| Citric acid monohydrate | 0.24 | mg/ml |
| Propyl gallate | 0.01 | mg/ml |
| Butylated hydroxytoluene | 0.05 | mg/ml |
| Trehalose | 250 | mg/ml |
| Water q.s. ad | 1 | ml |

at a pH of about 6.4.

In some embodiments, the formulation has ingredients comprising:

| Ingredient | Concentration | |
|---|---|---|
| Posaconazole | 50 | mg/ml |
| POPC | 40 | mg/ml |
| Histidine | 3 | mg/ml |
| Citric acid monohydrate | 0.24 | mg/ml |
| Alpha-D-tocopherol | 0.05 | mg/ml |
| Trehalose | 250 | mg/ml |
| Water q.s. ad | 1 | ml |

at a pH of about 6.5.

In some embodiments, the formulation has a wt. ratio of phospholipid to posaconazole between about 60:1 and about 1:10.

In some embodiments, the formulation has a wt. ratio of phospholipid to posaconazole between about 1:1 and about 1:5.

In some embodiments, the formulation has a wt. ratio of phospholipid to posaconazole between about 1:1 and about 4:5.

In some embodiments, the formulation has a the wt. ratio of thermoprotectant to posaconazole between about 300:1 and about 1:10.

In some embodiments, the formulation has a wt. ratio of thermoprotectant to posaconazole between about 1:1 and about 6:1.

In some embodiments, the formulation has a wt. ratio of thermoprotectant to phospholipid between about 30:1 and about 1:6.

In some embodiments, the formulation has a wt. ratio of thermoprotectant to phospholipid between about 5:4 and about 30:4.

In some embodiments, the invention encompasses a method of treating or preventing an infection inan animal in need thereof which comprises administering to said animal an effective amount of the formulation. In some embodiments, the animal is a mammal, a bird, a fish, or a reptile.

In some embodiments, the animal is a mammal, including but not limited to a human.

In some embodiments, the infection is caused by a fungus or a parasite.

In some embodiments, the infection is selected from the group consisting of:

oropharyngeal or esophageal candidiasis;

refractory oropharyngeal and esophageal candidiasis;

invasive aspergfilosis, candidiasis, fusariosis, scedosporiosis, infections due to dimorphic fungi, zygomycosis, and invasive infections due to rare molds and yeasts;

invasive mycoses in patients who are refractory to, or intolerant of, other therapies;

Candidiasis, invasive mould infections in patients who have undergone intensive chemotherapy and/or radiation therapy for hematologic malignancies, bone marrow or peripheral stem cell transplant conditioning regimens, and patients receiving combination immunosuppressive therapy for the treatment of acute or chronic graft-versus-host disease or prevention of solid organ transplantation;

Chagas disease; and,

Leishmaniasis.

In some embodiments, the invention encompasses a method wherein said formulation is administered intravenously.

In some embodiments, the invention encompasses a method wherein said formulation is administered intramuscularly, subcutaneously, ophthalmically, subconjuctivally, intraocularly, via anterior eye chamber injection, intravitreally, intraperitoneally, intrathecally, intracystically, intrapleurally, intranasally, topically, via wound irrigation, intradermally, intrabuccally, intra-abdominally, intra-articularly, intra-aurally, intrabronchially, intracapsularly, intrameningeally, intrapulmonarilly, via inhalation, via endotracheal or endobronchial installation, via direct installation into pulmonary cavities, intraspinally, intrasynovially, intrathoracically, via thoracostomy irrigation, vaginally, epidurally, rectally, intracistemally, intravascularly, intraventricularly, intraosseously, via irrigation of infected bone, or via application as part of any admixture with cement for prosthetic devices.

In some embodiments, the formulation further comprises a second active ingredient selected from one or more of the group consisting of: antifungals such as azoles; amphotericin B; deoxycholate amphotericin B; flucytosine; terbinafine; antibacterials; antivirals; steroids; nonsteroidal anti-inflammatory drugs (“NSAIDs”); chemotherapeutics; and anti-emitics.

In some embodiments, the invention encompasses a method further comprising administering a second active ingredient selected from one or more of the group consisting of: antifungals such as azoles; amphotericin B; deoxycholate amphotericin B; flucytosine; terbinafine; antibacterials; antivirals; steroids; nonsteroidal anti-inflammatory drugs (“NSAIDs”); chemotherapeutics; and, anti-emitics.

In some embodiments, the formulation is further characterized by providing a mean maximum plasma concentration (Cₘₐₓ) of posaconazole of at least about 467 ng/ml at steady state, and a mean plasma Area Under the Curve over 24 hours (AUC) value of posaconazole of at least about 9840 ng.hr/ml at steady state, when said formulation is infused over about 1 hour to deliver 100 mg of posaconazole, and repeated at an interval of about 24 hours.

In some embodiments, the formulation is further characterized by providing a mean maximum plasma concentration (Cₘₐₓ) of posaconazole of at least about 852 ng/ml at steady state, and a mean plasma Area Under the Curve over 24 hours (AUC) value of posaconazole of at least about 24,600 ng.hr/ml at steady state, when said formulation is infused over about 1 hour to deliver 200 mg of posaconazole, and repeated at an interval of about 24 hours.

In some embodiments, the formulation is further characterized by providing, after administration of a dosage of about 100 mg of said posaconazole, at least one of: a mean plasma half-life in a range of about 14.9 to about 38.4 hours; and a mean plasma steady state volume of distribution of about 200-500 L.

In some embodiments, the formulation is further characterized by providing a mean maximum plasma concentration (Cₘₐₓ) of posaconazole of at least about 1480 ng/ml at steady state, and a mean plasma Area Under the Curve over 24 hours (AUC) value of posaconazole of at least about 24,600 ng.hr/ml at steady state, when said formulation is infused over about 1 hour to deliver at least 200 mg of posaconazole, and repeated at an interval of about 24 hours.

In some embodiments, the formulation is further characterized as providing, after administration of a dosage of about 200 mg of said posaconazole, at least one of: a mean plasma half-life of about 18.7 to about 35.5 hours; and a mean plasma steady state volume of distribution of about 200-500 L.

In some embodiments, the formulation is further characterized as providing, after administration of a dosage of about 400 mg of said posaconazole, at least one of: a mean plasma half-life of about 18.5 to about 51.4 hours; and a mean plasma steady state volume of distribution of about 200-500 L.

In some embodiments, the formulation is further characterized as providing, after administration of a dosage of about 600 mg of said posaconazole, at least one of: a mean plasma half-life of about 27.2 to about 50.6 hours; and a mean plasma steady state volume of distribution of about 200-500 L.

In some embodiments, the formulation is further characterized as providing a mean posaconazole blood concentration profile substantially similar to that of FIG. 1, when said formulation is infused over about 1 hour to deliver 25-600 mg of posaconazole.

In some embodiments, the formulation is further characterized as providing a mean posaconazole plasma concentration profile substantially similar to that of FIG. 2, when said formulation is infused over about 1 hour to deliver 25-600 mg of posaconazole.

In some embodiments, the formulation is further characterized as providing a ratio of mean posaconazole blood Cₘₐₓ to mean posaconazole plasma Cₘₐₓ of between about 1.5 and about 3.8, when a single dose of said formulation is infused over about 1 hour to deliver 25-600 mg of posaconazole.

In some embodiments, the formulation is further characterized as providing a ratio of mean posaconazole blood Cₘₐₓ to mean posaconazole plasma Cₘₐₓ of between about 2.1 and about 3.3, when a single dose of said formulation is infused over about 1 hour to deliver 25 mg of posaconazole.

In some embodiments, the formulation is further characterized as providing a ratio of mean posaconazole blood Cₘₐₓ to mean posaconazole plasma Cₘₐₓ of between about 1.9 and about 3.8, when a single dose of said formulation is infused over about 1 hour to deliver 50 mg of posaconazole.

In some embodiments, the formulation is further characterized as providing a mean posaconazole blood Cₘₐₓ to mean posaconazole plasma Cₘₐₓ of between about 2.2 and about 3.3, when a single dose of said formulation is infused over about 1 hour to deliver 100 mg of posaconazole.

In some embodiments, the formulation is further characterized as providing a ratio of mean posaconazole blood Cₘₐₓ to mean posaconazole plasma Cₘₐₓ of between about 1.5 and about 3.2, when a single dose of said formulation is infused over about 1 hour to deliver 200 mg of posaconazole.

In some embodiments, the formulation is further characterized as providing a ratio of mean posaconazole blood Cₘₐₓ to mean posaconazole plasma Cₘₐₓ of between about 1.7 and about 3.3, when a single dose of said formulation is infused over about 1 hour to deliver 400 mg of posaconazole.

In some embodiments, the formulation is further characterized as providing a ratio of mean posaconazole blood Cₘₐₓ to mean posaconazole plasma Cₘₐₓ of between about 1.9 and about 3.1, when a single dose of said formulation is infused over about 1 hour to deliver 600 mg of posaconazole.

In some embodiments, the formulation is further characterized as providing a ratio of mean posaconazole blood Cₘₐₓ to mean posaconazole plasma Cₘₐₓ of between about 1.2 and about 2.5, at steady state when said formulation is infused over about 1 hour to deliver 25-600 mg of posaconazole, and repeated on a 24-hour basis.

In some embodiments, the formulation is further characterized as providing a ratio of mean posaconazole blood Cₘₐₓ to mean posaconazole plasma Cₘₐₓ of between about 1.5 and about 2.3, at steady state when said formulation is infused over about 1 hour to deliver 25 mg of posaconazole, and repeated on a 24-hour basis.

In some embodiments, the formulation is further characterized as providing a ratio of mean posaconazole blood Cₘₐₓ to mean posaconazole plasma Cₘₐₓ of between about 1.5 and about 2.4, at steady state when said formulation is infused over about 1 hour to deliver 50 mg of posaconazole, and repeated on a 24-hour basis.

In some embodiments, the formulation is further characterized as providing a ratio of mean posaconazole blood Cₘₐₓ to mean posaconazole plasma Cₘₐₓ of between about 1.7 and about 2.5, at steady state when said formulation is infused over about 1 hour to deliver 100 mg of posaconazole, and repeated on a 24-hour basis.

In some embodiments, the formulation is further characterized as providing a ratio of mean posaconazole blood Cₘₐₓ to mean posaconazole plasma Cₘₐₓ of between about 1.2 and about 2.0, at steady state when said formulation is infused over about 1 hour to deliver 200 mg of posaconazole, and repeated on a 24-hour basis.

In some embodiments, the formulation is further characterized as providing a ratio of mean posaconazole blood Cₘₐₓ to mean posaconazole plasma Cₘₐₓ of between about 1.2 and about 2.2, at steady state when said formulation is infused over about 1 hour to deliver 400 mg of posaconazole, and repeated on a 24-hour basis.

In some embodiments, the formulation is further characterized as providing a ratio of mean posaconazole blood Cₘₐₓ to mean posaconazole plasma Cₘₐₓ of between about 1.3 and about 1.7, at steady state when said formulation is infused over about 1 hour to deliver 600 mg of posaconazole, and repeated on a 24-hour basis.

In some embodiments, the water in the formulation has been removed by lyophilization.

In some embodiments, the animal treated is human, while in other embodiments the animal treated is non-human.

In some embodiments, the formulation is one that is bioequivalent to a formulation disclosed herein.

In some embodiments, the method further comprises administering a bolus loading dose of said formulation and then administering an intravenous maintenance dose of said formulation.

In some embodiments, the method comprises administering to said animal an effective amount of posaconazole to provide a mean maximum plasma concentration (Cₘₐₓ) of posaconazole of at least about 467 ng/ml at steady state, and a mean plasma Area Under the Curve over 24 hours (AUC) value of posaconazole of at least about 9840 ng.hr/ml at steady state, when said formulation is infused over about 1 hour to deliver 100 mg of posaconazole, and repeated at an interval of about 24 hours.

### BRIEF DESCRIPTION OF THE DRAWINGS

FIG. 1 shows posaconazole mean blood concentration-time profiles in healthy volunteers after 1 hr intravenous infusions of 25, 50, 100, 200, 400, and 600 mg posaconazole.

FIG. 2 shows posaconazole mean plasma concentration-time profiles in healthy volunteers after 1 hr intravenous infusions of 25, 50, 100, 200, 400, and 600 mg posaconazole.

FIG. 3 shows posaconazole mean plasma and blood concentration-time profiles in healthy volunteers after 1 hr intravenous infusion of 25 mg posaconazole.

FIG. 4 shows posaconazole mean plasma and blood concentration-time profiles in healthy volunteers after 1 hr intravenous infusion of 600 mg posaconazole.

### DETAILED DESCRIPTION OF THE INVENTION

The present invention encompasses formulations suitable for parenteral administration, e.g., by injection, for treating an infection. These formulations comprise a suspension of posaconazole, stabilized by a phospholipid, in a mixture comprising water, a thermoprotectant, and a buffer system. Since posaconazole is minimally soluble in water, a suspension formulation is advantageous. Phospholipids have been found to be effective surfactants in forming stable suspensions of posaconazole in water or an aqueous medium.

These phospholipids can degrade when subjected to the temperature excursions experienced during terminal sterilization (e.g., autoclaving), a step which is necessary to assure the sterility of any injectable formulation. Thus, a thermoprotectant is used to prevent agglomeration and crystal growth of the posaconazole particles during autoclaving.

Parenteral buffer systems are typically designed to be at physiological pH of about 7.4. Phospholipids are known to be stable at a pH range of about 6 to about 7. Furthermore, pH adjustment of injectable formulations can be necessary to achieve physiological compatibility, and thus, for example, to minimize injection-site irritation. In addition, the rate of phospholipid hydrolysis can be temperature-sensitive. Thus, in the present formulations, the buffer systems are designed to meet physiological pH requirements, and to maintain the temperature/pH-dependent chemical stability of the phospholipid in the formulation during high temperature excursions (such as experienced during autoclaving), and throughout shelf life.

In accordance with the above, it was found that POPC, an ingredient that acts as a suspension stabilizer, was sensitive to autoclaving. Certain buffer systems were found to control degradation of POPC-containing posaconazole formulations during autoclaving. For example, such formulations were found to be stable after 20 minutes of autoclaving at 121° C. In addition, these buffer systems stabilize such formulations during storage at 4° C. to 25° C. for at least 18 months following autoclaving. Similarly, other phospholipids that are similar to POPC could be used to stabilize the formulations disclosed herein. For example, unsaturated phospholipids with an acyl chain length ranging from C₁₂ to C₂₀ wherein the degree of unsaturation of the acyl chain ranges from 1 to 4; as well as saturated phospholipids with an acyl chain length ranging from C₁₂ to C₁₈ are useful according to the present invention. Examples of useful unsaturated phospholipids include: 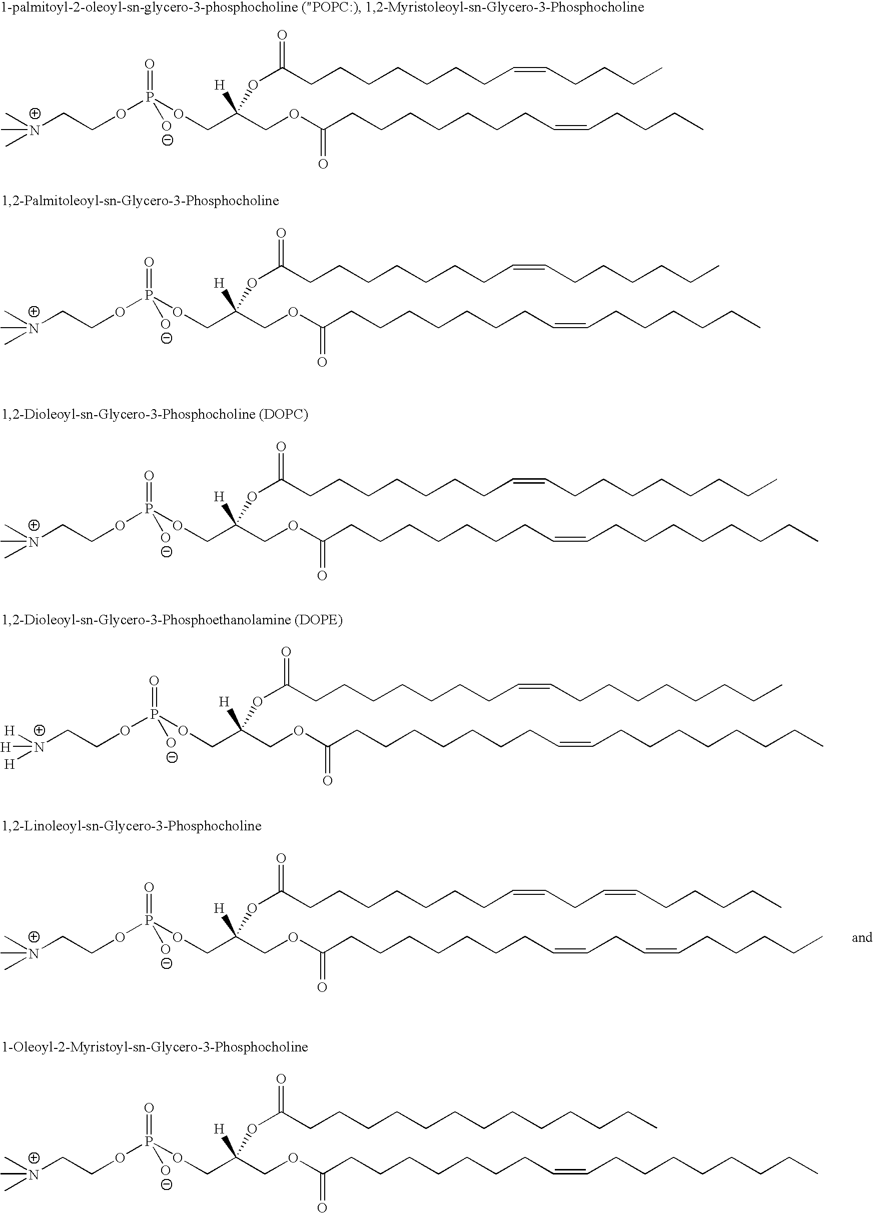
or combinations thereof

Examples of saturated phospholipids include: 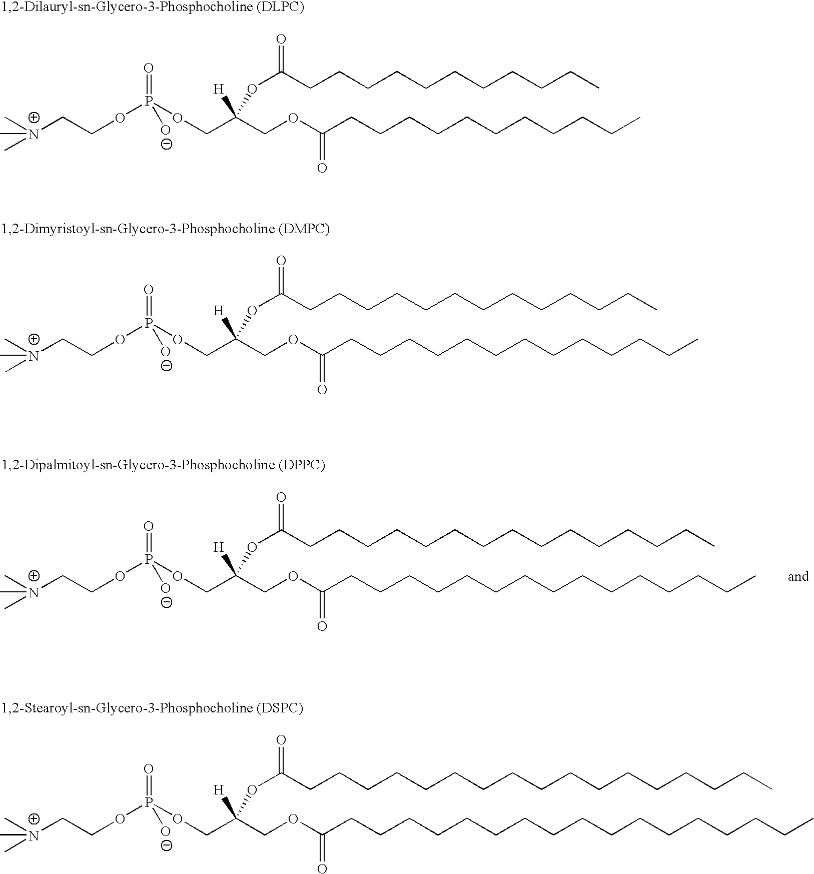
or combinations thereof.

Unsaturated phospholipids are known be to prone to oxidation. To prevent such oxidation, an antioxidant can be employed. In some embodiments, the antioxidant comprises propyl gallate, preferably at a concentration of about 0.02 to about 0.005 mg/ml. In other embodiments, the antioxidant comprises butylated hydroxytoluene, preferably at a concentration of about 0.1 to about 0.02 mg/ml. In related embodiments, the antioxidant comprises propyl gallate, preferably at a concentration of about 0.02 to about 0.005 mg/ml, in combination with butylated hydroxytoluene, preferably at a concentration of about 0.1 to about 0.02 mg/ml. In yet other embodiments, the antioxidant comprises alpha-D-tocopherol, preferably at a concentration of about 0.5 to about 0.01 mg/ml.

The inventors have found certain ratios of components to result in advantageous formulations. For example, the weight ratio of phospholipid to posaconazole is preferably between about 1:0.1 and about 1:10, more preferably, between about 1:1 and about 1:5, still more preferably, between about 1:1 and about 4:5. The weight ratio of thermoprotectant to posaconazole is preferably between about 0.5:1 and about 6:1, more preferably, between about 2:1 and about 6:1. The weight ratio of thermoprotectant to phospholipid is preferably, between about 20:1 and about 5:4, more preferably, between about 20:4 and about 30:4.

The formulations of the present invention comprise a suspension of solid particles of posaconazole of specific particle size distribution in an aqueous phase. The particle size distribution displayed in the suspended particles is critical for physiological compatibility, syringeability, physical stability of the suspension, re-suspendability, and for pharmacokinetic characteristics and bio-distribution (i.e., sequestration within specific bodily tissues). Since these characteristics are critical to the formulation as delivered to the patient, it is important that processes that contribute to changes in particle size distribution after micronization are controlled.

Such processes can include agglomeration during autoclaving, and de-suspension due to temperature excursions and/or agitation experienced during shipping and storage. It is the particle size distribution in the formulation as ready for administration to the patient that influences pharmacokinetic characteristics and bio-distribution.

The inventors of the present invention have determined that for injectable formulations of posaconazole, these characteristics are brought within advantageous ranges with particle size distributions whose median values are between about 1.0 to about 8.0 microns, preferably, between about 1.0 to about 5.0 microns, more preferably between about 1.2 to about 4.5 microns. In each case, the particle size distributions display not more than about 3000 particles of 10 microns or greater size and not more than about 300 particles of 25 microns or greater size.

In the injectable formulations of the present invention, which include POPC, it has been found useful to maintain a pH range of between about 3.0 and about 9.0, preferably between about 6.0 and about 8.0, and more preferably between about 6.4 and about 7.6.

The inventors have found that certain organic buffers, e.g., histidine and citric acid, are more advantageous in controlling the pH-related degradation of POPC in the formulation. Components used in pH adjustment systems can also function as components of the buffer system, after pH adjustment has been achieved. Non-limiting examples of pH adjustment system components that function in this way include sodium hydroxide, hydrochloric acid, and phosphoric acid.

Anti-Infective Applications

The present invention encompasses methods of prevention and treatment of a variety of infections caused by a broad spectrum of infectious agents. The term “infection” is understood to include, but not be limited to, those disease states caused by molds, yeasts and other infectious agents, such as: Candida, dermatophytes, Dimorphics, Dematiaceous (e.g., Alternaria and Bipolaris), Aspergillus, Acremonium, Basidiomycetes, Bjerkandera, Coprinus, Paecilomyces, Microsporum, Trichophyton, Pseudallescheria, Schizophyllum, Crytococcus, Histoplasma, Blastomyces, Coccidioides, Fusarium, Exophiala, Zygomycocetes (e.g., Mucor, Rhizopus, and Rhizomucor), Kluyveromyces, Saccharomyces, Yarrowia, Pichia, Epidermophyton, Paracoccidioides, Scedosporium, Apophysomyces, Curvularia, Penicillium, Fonsecaea, Wangiella, Sporothrix, Pneumocystis, Trichosporon, Absidia, Cladophialophora, Ramichloridium, Syncephalastrum, Madurella, Scytalidium, Leshmania, protozoa, bacteria, gram negatives, gram positives, anaerobes, including Legionella Borrelia, Mycoplasma, Treponema, Gardneralla, Trichomononas and Trypanosoma.

The present invention is intended to treat both opportunistic and non-opportunistic infections, where the term “opportunistic” as used herein denotes those infections caused by organisms capable of causing a disease only in a host whose resistance is lowered, e.g., by chemotherapy or H.I.V.

In particular, posaconazole is useful in the prevention and/or treatment of the following disease states:

Initial (first line) treatment of oropharyngeal or esophageal candidiasis;

Salvage therapy of azole-refractory oropharyngeal and esophageal candidiasis (e.g., in patients who have failed oral fluconazole and/or itraconazole);

Initial treatment of invasive aspergillosis, candidiasis, fusariosis, scedosporiosis, infections due to dimorphic fungi (e.g., cryptococcosis, coccidioidomycosis, paracoccidioidomycosis, histoplasmosis, blastomycosis), zygomycosis, and invasive infections due to rare moulds and yeasts;

Salvage therapy for invasive mycoses in patients who are refractory to or intolerant of other therapies (e.g., amphotericin B, lipid formulations of amphotericin B, caspofungin, voriconazole and/or itraconazole);

Prevention of invasive Candidiasis, invasive mould infections (including zygomycosis and aspergillosis) in patients at high risk, including patients who have undergone intensive chemotherapy and/or radiation therapy for hematologic malignancies, bone marrow or peripheral stem cell transplant conditioning regimens, and patients receiving combination immunosuppressive therapy for the treatment of acute or chronic graft-versus-host disease or prevention of solid organ transplantation;

Chagas disease (Trypanosomiasis due to T. cruzi) including acute and chronic forms; and,

Leishmaniasis, including visceral and localized forms.

Administration

Immuno-suppressant therapy (e.g., chemotherapy, radiation therapy, myeloablative conditioning regimens) often results in one or more of the above-referenced infections. The present invention encompasses the administration of a posaconazole formulation adjunctive to immuno-suppressant therapy, wherein the posaconazole formulation functions prophylactically with regard to opportunistic infections including the above-referenced disease states.

The present invention encompasses a variety of modes of administration to any part, organ, interstice or cavity ofan animal's body that is subject to an infection. A non-limiting set of examples of modes by which the posaconasole formulations of the present invention may be administered includes: intravenously, intramuscularly, subcutaneously, ophthalmically, subconjuctivally, intraocularly, via anterior eye chamber injection, intravitreally, intraperitoneally, intrathecally, intracystically, intrapleurally, intranasally, topically, via wound irrigation, intradermally, intrabuccally, intra-abdominally, intra-articularly, intra-aurally, intrabronchially, intracapsularly, intrameningeally, intrapulmonarilly, via inhalation, via endotracheal or endobronchial installation, via direct installation into pulmonary cavities, intraspinally, intrasynovially, intrathoracically, via thoracostomy irrigation, vaginally, epidurally, rectally, intracisternally, intravascularly, intraventricularly, intraosseously, via irrigation of infected bone, and via application as part of any admixture with cement for prosthetic devices.

Co-formulations comprising combinations of posaconazole and at least one other active ingredient are also within the scope of the present invention. Non-limiting examples of such active ingredients include: antifungals such as echinocandins (including caspofungin, micafungin, and anidulafungin) and azoles (including voriconazole, itraconazole, fluconazole, ketoconazole, ravuconazole); amphotericin B; deoxycholate amphotericin B; flucytosine; and terbinafine.

Also within the scope of this invention are combinations with an antibacterial, antiviral, steroid, or nonsteroidal anti-inflammatory drugs (“NSAIDs”), chemotherapeutics, and/or anti-emitics. Similarly, co-administration of posaconazole with at least one of the above active ingredients, aside from within a single formulation, is also within the scope of the present invention.

Also within the scope of the present invention are a variety of dosing regimens, each consisting of a frequency of dosing and a duration of administration. Preferred frequencies of dosing include once every 12, 24, 36 and 48 hours. Preferred durations of administration are within the range of 30 minutes to 4 hours, more preferably, 1 to 2 hours. Also included within the scope of preferred administration is bolus dosing, at various rates and various doses, and combinations of a bolus loading dose, or several bolus loading doses, with an intravenous infusion maintenance dose that provides therapeutic plasma concentration ranges similar to or exceeding those described in Table 14 infra.

As used herein, the following terms shall have the definitions set forth below.

As used herein, the phrase “phospholipid” refers to a lipid compound that yields on hydrolysis phosphoric acid, an alcohol, fatty acid and a nitrogenous base. Examples include natural and synthetic phoshpholipids, which include lecithin, cephalin, sphingomyelin and 1-palmitoyl-2-oleoyl-sn-glycero-3-phosphocholine (“POPC”).

As used herein, the phrase “natural phospholipid” refers to a phospholipid occurring in nature, or derived from a natural source. Non-limiting examples of natural phospholipids include egg phospholipids, soy phospholipids, and animal tissue phospholipids. Combinations of more than one natural phospholipid are within the scope of the present invention.

As used herein, the phrase “synthetic phospholipid” refers to a man-made phospholipid. Non-limiting examples of synthetic phospholipids include 1-palmitoyl-2-oleoyl-sn-glycero-3-phosphocholine (POPC), 1,2-oleoyl-sn-glycero-3-phosphocholine (DOPC), 1,2-Dilauryl-sn-Glycero-3-Phosphocholine (DLPC), 1,2-Dimyristoyl-sn-Glycero-3-Phosphocholine (DMPC), 1,2-Dipalmitoyl-sn-Glycero-3-Phosphocholine (DPPC) and 1,2-Stearoyl-sn-Glycero-3-Phosphocholine (DSPC). Combinations of more than one synthetic phospholipid are within the scope of the present invention.

As used herein, the phrase “buffer system” refers to a buffer comprising one or more components that maintains a particular pH range. Non-limiting examples of suitable buffer systems include: phosphoric acid; glycine; sodium citrate; histidine; citric acid; acetic acid; tromethamine; ammonium sulfate; and combinations thereof. The aforementioned components are understood to include the salts, hydrates and solvates thereof. Thus, for example, phosphoric acid includes the sodium phosphate or potassium phosphate salts, among other salts. Preferred buffer systems include sodium phosphate monobasic, sodium phosphate dibasic, or a combination thereof. More preferred buffer systems include sodium phosphate monobasic monohydrate, sodium phosphate dibasic anhydrous, or a combination thereof. As used herein, the phrase “organic buffer” refers to a buffer comprising at least one organic compound. Non-limiting examples of suitable organic buffers include: glycine; sodium citrate; histidine; citric acid; acetic acid; and combinations thereof.

As used herein, the term “antioxidant” refers to an agent that hinders oxidation. Exemplary antioxidants include propyl gallate, butylated hydroxytoluene, and alpha-D-tocopherol.

As used herein, the phrase “median particle size” refers to the particle size present in the volume-weighted 50^{th} percentile, as ascertained by Malvern®, Sympatec®, or Horibe® laser diffraction particle size analysis. Particle sizes are measured throughout, and at the termination of, the shelf life, typically up to 24 months after manufacture, when held at either refrigerated or room temperatures. Particle sizes are also measured and maintained when the formulation is diluted into large volume parenterals, e.g., 5% dextrose or water for injection.

As used herein, the phrase “initial median particle size” refers to the particle size present within 1 week after a specified timepoint. For example, the initial median particle size after autoclaving refers to the median particle size present within 1 week after autoclaving has been completed.

As used herein, the term “autoclaving” refers to sterilization by the terminal steam sterilization method. For example, autoclaving for 20 minutes at 121° C. suffices to sterilize the posaconazole formulations disclosed herein.

As used herein, the phrase “thermoprotectant” refers to an agent that stabilizes the phospholipid during temperature excursions. In the present invention, a thermoprotectant is used to preserve the phospholipid, which is necessary to control crystal growth and aggolomeration of the posaconazole particles during autoclaving. Thermoprotectants are typically water soluble polyhydroxyl compounds. For example, trehalose is a thermoprotectant agent that may be used in conjunction with posaconazole. Others include maltose, sorbitol, dextrose, sucrose, lactose and mannitol.

As used herein, the term “prodrug” refers to a compound that is a drug precursor which, upon administration to a subject, undergoes chemical conversion by metabolic or chemical processes to yield posaconazole or a salt and/or solvate thereof.

As used herein, the term “solvate” refers to a physical association between a compound with one or more solvent molecules. This physical association involves varying degrees of ionic and/or covalent bonding, including hydrogen bonding. In certain instances, the solvate will be capable of isolation, for example, when one or more solvent molecules are incorporated in the crystal lattice of the crystalline solid. The term “solvate” encompasses both solution-phase and isolatable solvates. Non-limiting examples of suitable solvates include hydrates, ethanolates, and methanolates.

As used herein, the term “injectable” means adapted to parenteral administration.

As used herein, the term “fungus” means one of the diverse morphologic forms of yeasts and molds. Fungi include Candida, dermatophytes, Dimorphics, Dematiaceous (e.g., Altemaria and Bipolaris), Aspergillus, Acremonium, Basidiomycetes, Bjerkandera, Coprinus, Paecilomyces, Microsporum, Trichophyton, Pseudallescheria, Schizophyllum, Crytococcus, Histoplasma, Blastomyces, Coccidioides, Fusarium, Exophiala, Zygomycocetes (e.g., Mucor, Rhizopus, and Rhizomucor), Kluyveromyces, Saccharomyces, Yarrowia, Pichia, Epidermophyton, Paracoccidioides, Scedosporium, Apophysomyces, Curvularia, Penicillium, Fonsecaea, Wangiella, Sporothrix, Pneumocystis, Trichosporon, Absidia, Cladophialophora, Ramichloridium, Syncephalastrum, Madurella, Scytalidium, Leshmania, gram negatives, gram positives, Mycoplasma, Treponema, Gardneralla, and Trichomononas.

As used herein, the term “Dematiaceous” means dark conidia and/or hyphae, and includes as non-limiting examples Altemaria and Bipolaris.

As used herein, the term “Zygomycocete” means a class of fungi characterized by sexual reproduction resulting in the formation of zygospore, and asexual reproduction by means of nonmotile spores called sporangiospores or conidia, and includes as non-limiting examples Mucor, Rhizopus, and Rhizomucor.

As used herein, the term “anaerobe” means a microorganism that can live and grow in the absence of oxygen, and includes as non-limiting examples Legionella Borrelia, Mycoplasma, Treponema, Gardneralla, and Trichomononas.

As used herein, the term “parasite” means an organism that lives on or in another and draws its nourishment therefrom. Parasites include Leshmania and Trypansoma, among others.

As used herein, the term “antifungal” means an agent having activity against one or more fungi, and includes echinocandins such as caspofungin, micafungin, and anidulafungin.

As used herein, the term “azole” means divinylenimine, and includes voriconazole, itraconazole, fluconazole, ketoconazole, ravuconazole.

As used herein, the term “mean maximum concentration (Cₘₐₓ)” when followed by the term “at steady state” means that mean maximum concentration value that occurs after administration of a sufficient number of repeated doses of the formulation to generate maximum blood or plasma concentrations that are substantially equivalent to one another in value. Thus, the subsequent maximum concentration values are no longer rising, but rather each peak achieves substantially the same maximum value as the previous one and the next one.

As used herein, the term “animal” is understood to include humans, non-human mammals, fish, birds and reptiles.

As used herein, the term “bioequivalent” is understood as having that meaning assigned to the term by the U.S. Food & Drug Administration. “Bioequivalence means the absence of a significant difference in the rate and extent to which the active ingredient or active moiety in pharmaceutical equivalents or pharmaceutical alternatives becomes available at the site of drug action when administered at the same molar dose under similar conditions in an appropriately designed study.” 21 CFR 320.1(e). Methodologies for determining bioequivalence are given in “Guidance for Industry: Statistical Approaches to Establishing Bioequivalence,” U.S. Department of Health and Human Services, Food and Drug Administration, Center for Drug Evaluation and Research (CDER) June, 2001.

### EXAMPLES

The following non-limiting examples illustrate certain aspects of the invention.

Exemplary formulations of posaconazole in conjunction with POPC and trehalose using various buffer systems are detailed below in Tables 1-3. These formulations provide ranges for buffer systems that maintain a particular pH range.

TABLE 1
Representative posaconazole formulations at a pH range of 6.4-7.4

| Function | Ingredient | Concentration range | |
|---|---|---|---|
| Active | Posaconazole | 50 | mg/ml |
| Stabilizer | POPC | 40 | mg/ml |
| Buffer | Glycine | 3.5-10.5 | mg/ml |
| Buffer | Sodium citrate dihydrate | 4-10.2 | mg/ml |
| Buffer | Citric acid monohydrate | 0.01-0.02 | mg/ml |
| Stabilizer | Trehalose | 250 | mg/ml |
| Solvent | Water q.s. ad | 1 | ml |

TABLE 2
Representative posaconazole formulations at a pH range of 6.4-6.6

| Function | Ingredient | Concentration range | |
|---|---|---|---|
| Active | Posaconazole | 50 | mg/ml |
| Stabilizer | POPC | 40 | mg/ml |
| Buffer | Glycine | 1.5-4.5 | mg/ml |
| Buffer | Citric acid monohydrate | 0.12-0.36 | mg/ml |
| Stabilizer | Trehalose | 250 | mg/ml |
| Solvent | Water q.s. ad | 1 | ml |

TABLE 3
Representative posaconazole formulations at a pH range of 6.6-6.8

| Function | Ingredient | Concentration range | |
|---|---|---|---|
| Active | Posaconazole | 50 | mg/ml |
| Stabilizer | POPC | 40 | mg/ml |
| Buffer | Histidine | 1.5-4.5 | mg/ml |
| Buffer | Ammonium sulfate | 1-3 | mg/ml |
| Buffer | Hydrochloric acid | 0.1-0.3 | mg/ml |
| Stabilizer | Trehalose | 250 | mg/ml |
| Solvent | Water q.s. ad | 1 | ml |

An exemplary posaconazole formulation for each of the buffer systems described in Tables 1-3 is provided in Examples 1-3, respectively.

### Example 1

| Ingredient | Concentration | |
|---|---|---|
| Posaconazole | 50 | mg/ml |
| POPC | 40 | mg/ml |
| Glycine | 7 | mg/ml |
| Sodium citrate dihydrate | 8 | mg/ml |
| Trehalose | 250 | mg/ml |
| Water q.s. ad | 1 | ml |

Of note, the pH is 7.4 in Example 1.

### Example 2

| Ingredient | Concentration | |
|---|---|---|
| Posaconazole | 50 | mg/ml |
| POPC | 40 | mg/ml |
| Histidine | 3 | mg/ml |
| Citric acid monohydrate | 0.24 | mg/ml |
| Trehalose | 250 | mg/ml |
| Water q.s. ad | 1 | ml |

Of note, the pH is 6.4 in Example 2.

### Example 3

| Ingredient | Concentration | |
|---|---|---|
| Posaconazole | 50 | mg/ml |
| POPC | 40 | mg/ml |
| Histidine | 3 | mg/ml |
| Ammonium sulfate | 2 | mg/ml |
| Hydrochloric acid | 0.2 | mg/ml |
| Trehalose | 250 | mg/ml |
| Water q.s. ad | 1 | ml |

Of note, the pH is 6.6 in Example 3.

In addition, exemplary posaconazole formulations that include antioxidant are described in Examples 4-6.

### Example 4

| Ingredient | Concentration | |
|---|---|---|
| Posaconazole | 50 | mg/ml |
| POPC | 40 | mg/ml |
| Glycine | 7 | mg/ml |
| Sodium citrate dihydrate | 8 | mg/ml |
| Propyl gallate | 0.01 | mg/ml |
| Butylated hydroxytoluene | 0.05 | mg/ml |
| Trehalose | 250 | mg/ml |
| Water q.s. ad | 1 | ml |

Of note, the pH is 7.4 in Example 4.

### Example 5

| Ingredient | Concentration | |
|---|---|---|
| Posaconazole | 50 | mg/ml |
| POPC | 40 | mg/ml |
| Histidine | 3 | mg/ml |
| Citric acid monohydrate | 0.24 | mg/ml |
| Propyl gallate | 0.01 | mg/ml |
| Butylated hydroxytoluene | 0.05 | mg/ml |
| Trehalose | 250 | mg/ml |
| Water q.s. ad | 1 | ml |

The pH is 6.4 in Example 5.

### Example 6

| Ingredient | Concentration | |
|---|---|---|
| Posaconazole | 50 | mg/ml |
| POPC | 40 | mg/ml |
| Histidine | 3 | mg/ml |
| Citric acid monohydrate | 0.24 | mg/ml |
| Alpha-D-tocopherol | 0.05 | mg/ml |
| Trehalose | 250 | mg/ml |
| Water q.s. ad | 1 | ml |

The pH is 6.5 in Example 6.

Example 7 is a preferred embodiment of the present invention.

### Example 7

| Ingredient | Concentration | |
|---|---|---|
| Posaconazole | 50 | mg/ml |
| POPC | 40 | mg/ml |
| Sodium Phosphate, Monobasic, | 0.345 | mg/ml |
| Monohydrate, USP | | |
| Sodium Phosphate, Dibasic, | 1.065 | mg/ml |
| Anhydrous, USP | | |
| Trehalose | 250 | mg/ml |
| Sodium Hydroxide, NF (1.0 N) | for pH adjustment | |
| Phosphoric Acid, NF (20% w/w) | for pH adjustment | |
| Water for injection, USP | qs 1 | ml |

The pH is 7.2 in Example 7.

The following is an exemplary placebo formulation wherein the pH is 6.4. This exemplary placebo formulation was utilized in the comparative stability data study described below.

| Ingredient | Concentration | |
|---|---|---|
| Placebo | | |
| POPC | 40 | mg/ml |
| Glycine | 1 | mg/ml |
| Sodium citrate dihydrate | 0.3 | mg/ml |
| Citric acid monohydrate | 0.016 | mg/ml |
| Trehalose | 250 | mg/ml |
| Water q.s. ad | 1 | ml |

Comparative Stability Data Study

The stability of POPC in formulation Examples 1-3 was compared with the aforementioned exemplary placebo both before and after autoclaving for 20 min at 121° C. In addition, posaconazole stability, particle size, pH, and a physical observation were ascertained for each formulation before and after autoclaving. Each formulation was also examined following an additional period of storage at 4° C., 25° C., and 40° C. (i.e., 4° C.±2° C. at 60%±5% relative humidity; 25° C.±2° C. at 60%±5% relative humidity; and 40° C.±2° C. at ambient relative humidity, respectively) for 1 month, 3 months, and 6 months after autoclaving. Notably, particle size was determined using the Malvern laser diffraction particle size analysis technique. Particle sizes are characterized by values for median (“50^{th} percentile”) and maximum (“100^{th} percentile”). The stability data from these comparative studies are compiled below for formulations reflected in Examples 1-6, shown in Tables 4-9, respectively.

TABLE 4
Stability data for posaconazole formulation Example 1

| | | | | Particle size | | | |
|---|---|---|---|---|---|---|---|
| | | | | (microns) | | | |
| Interval/ | | Posaconazole | POPC | 50^{th} | 100^{th} | | Physical |
| Condition | | (mg/ml) | (mg/ml) | percentile | percentile | pH | Observation |
| Initial - | | 51.3 | 41.4 | 1.11 | 3.77 | 7.4 | Milky white |
| Before | | | | | | | |
| Autoclaving | | | | | | | |
| Initial - | | 50.8 | 40.9 | 1.49 | 6.63 | 7.3 | Milky white |
| After | | | | | | | |
| Autoclaving | | | | | | | |
| 1 | 4° C. | 50.2 | 39.0 | 1.48 | 5.49 | 7.2 | Milky white |
| month | 25° C. | 50.7 | 39.2 | 1.48 | 5.49 | 7.2 | Milky white |
| | 40° C. | 50.6 | 39.1 | 1.49 | 5.49 | 7.1 | Milky white |
| 3 | 4° C. | 55.1 | 41.8 | 1.47 | 5.49 | 7.2 | Milky white |
| months | 25° C. | 55.4 | 41.5 | 1.48 | 6.63 | 7.2 | Milky white |
| | 40° C. | 55.4 | 40.0 | 1.49 | 6.63 | 7.1 | Milky white |
| 6 | 4° C. | 51.7 | 44.4 | 1.44 | 4.88 | 7.3 | Milky white |
| months | 25° C. | 50.3 | 42.3 | 1.50 | 5.69 | 7.3 | Milky white |
| | 40° C. | 51.5 | 36.0 | 1.57 | 5.69 | 7.3 | Milky white |

TABLE 5
Stability data for posaconazole formulation Example 2

| | | | | Particle size | | | |
|---|---|---|---|---|---|---|---|
| | | | | (microns) | | | |
| Interval/ | | Posaconazole | POPC | 50^{th} | 100^{th} | | Physical |
| Condition | | (mg/ml) | (mg/ml) | percentile | percentile | pH | Observation |
| Initial - | | 46.4 | 38.2 | 1.41 | 6.63 | 6.4 | Milky white |
| Before | | | | | | | |
| Autoclaving | | | | | | | |
| Initial - | | 46.3 | 38.2 | 1.76 | 6.63 | 6.4 | Milky white |
| After | | | | | | | |
| Autoclaving | | | | | | | |
| 1 | 4° C. | 45.8 | 37.9 | 1.70 | 6.63 | 6.4 | Milky white |
| month | 25° C. | 45.3 | 37.0 | 1.70 | 6.63 | 6.4 | Milky white |
| | 40° C. | 45.8 | 37.4 | 1.72 | 6.63 | 6.4 | Milky white |
| 3 | 4° C. | 44.8 | 36.1 | 1.69 | 6.63 | 6.4 | Milky white |
| months | 25° C. | 45.9 | 36.8 | 1.70 | 6.63 | 6.4 | Milky white |
| | 40° C. | 45.6 | 35.7 | 1.76 | 35.98 | 6.4 | Milky white |
| 6 | 4° C. | 44.1 | 38.8 | 1.65 | 6.63 | 6.6 | Milky white |
| months | 25° C. | 46.1 | 40.1 | 1.71 | 6.63 | 6.6 | Milky white |
| | 40° C. | 46.1 | 40.1 | 1.70 | 6.63 | 6.6 | Milky white |

TABLE 6
Stability data for posaconazole formulation Example 3

| | | | | Particle size | | | |
|---|---|---|---|---|---|---|---|
| | | | | (microns) | | | |
| Interval/ | | Posaconazole | POPC | 50^{th} | 100^{th} | | Physical |
| Condition | | (mg/ml) | (mg/ml) | percentile | percentile | pH | Observation |
| Initial - | | 46.1 | 36.7 | 1.39 | 5.49 | 6.6 | Milky white |
| Before | | | | | | | |
| Autoclaving | | | | | | | |
| Initial - | | 45.9 | 36.2 | 1.75 | 6.63 | 6.6 | Milky white |
| After | | | | | | | |
| Autoclaving | | | | | | | |
| 1 | 4° C. | 45.3 | 34.6 | 1.76 | 6.63 | 6.5 | Milky white |
| month | 25° C. | 44.9 | 34.4 | 1.76 | 6.63 | 6.5 | Milky white |
| | 40° C. | 44.9 | 34.5 | 1.75 | 6.63 | 6.5 | Milky white |
| 3 | 4° C. | 46.9 | 35.0 | 1.77 | 6.63 | 6.5 | Milky white |
| months | 25° C. | 46.9 | 34.9 | 1.78 | 6.63 | 6.5 | Milky white |
| | 40° C. | 47.5 | 34.5 | 1.75 | 6.63 | 6.5 | Milky white |
| 6 | 4° C. | 47.0 | 35.3 | 2.18 | 6.63 | 6.6 | Milky white |
| months | 25° C. | 46.3 | 35.1 | 1.75 | 5.69 | 6.5 | Milky white |
| | 40° C. | 49.4 | 32.5 | 2.03 | 6.63 | 6.6 | Milky white |

TABLE 7
Stability data for posaconazole formulation Example 4

| | | | | Particle size | | | |
|---|---|---|---|---|---|---|---|
| | | | | (microns) | | | |
| Interval/ | | Posaconazole | POPC | 50^{th} | 100^{th} | | Physical |
| Condition | | (mg/ml) | (mg/ml) | percentile | percentile | pH | Observation |
| Initial - | | 65.4 | 50.3 | 1.21 | 5.49 | 7.3 | Milky white |
| Before | | | | | | | |
| Autoclaving | | | | | | | |
| Initial - | | 65.5 | 50.2 | 1.66 | 6.63 | 7.2 | Milky white |
| After | | | | | | | |
| Autoclaving | | | | | | | |
| 1 | 4° C. | 65.2 | 50.2 | 1.65 | 6.63 | 7.2 | Milky white |
| month | 25° C. | 65.1 | 50.4 | 1.64 | 6.63 | 7.2 | Milky white |
| | 40° C. | 67.1 | 50.6 | 1.67 | 6.63 | 7.2 | Milky white |
| 3 | 4° C. | 68.1 | 50.9 | 1.64 | 6.63 | 7.4 | Milky white |
| months | 25° C. | 68.4 | 51.0 | 1.64 | 6.63 | 7.4 | Milky white |
| | 40° C. | 69.5 | 49.2 | 1.67 | 29.82 | 7.3 | Milky white |
| 6 | 4° C. | 66.7 | 53.3 | 1.61 | 5.68 | 7.2 | Milky white |
| months | 25° C. | 64.9 | 52.6 | 1.54 | 4.88 | 7.0 | Milky gray |
| | 40° C. | 65.4 | 47.3 | 1.64 | 56.23 | 6.8 | Milky white |

TABLE 8
Stability data for posaconazole formulation Example 5

| | | | | Particle size | | | |
|---|---|---|---|---|---|---|---|
| | | | | (microns) | | | |
| Interval/ | | Posaconazole | POPC | 50^{th} | 100^{th} | | Physical |
| Condition | | (mg/ml) | (mg/ml) | percentile | percentile | pH | Observation |
| Initial - | | 50.8 | 39.7 | 1.61 | 6.63 | 6.5 | Milky white |
| After | | | | | | | |
| Autoclaving | | | | | | | |
| 1 | 4° C. | 50.9 | 39.6 | 1.61 | 6.63 | 6.5 | Milky white |
| month | 25° C. | 51.1 | 39.8 | 1.60 | 6.63 | 6.5 | Milky white |
| | 40° C. | 50.7 | 39.4 | 1.62 | 6.63 | 6.5 | Milky white |
| 3 | 4° C. | 53.0 | 40.5 | 1.62 | 6.63 | 6.6 | Milky white |
| months | 25° C. | 53.1 | 40.6 | 1.62 | 6.63 | 6.6 | Milky white |
| | 40° C. | 51.9 | 39.0 | 1.62 | 6.63 | 6.7 | Milky white |
| 6 | 4° C. | 54.2 | 46.2 | 1.59 | 5.69 | 6.5 | Milky white |
| months | 25° C. | 53.2 | 44.6 | 1.59 | 5.69 | 6.5 | Milky white |
| | 40° C. | 52.2 | 41.2 | 1.58 | 5.69 | 6.5 | Milky white |

TABLE 9
Stability data for posaconazole formulation Example 6

| | | | | Particle size | | | |
|---|---|---|---|---|---|---|---|
| | | | | (microns) | | | |
| Interval/ | | Posaconazole | POPC | 50^{th} | 100^{th} | | Physical |
| Condition | | (mg/ml) | (mg/ml) | percentile | percentile | pH | Observation |
| Initial - | | 46.8 | 36.5 | 1.32 | 6.63 | 6.5 | Milky white |
| Before | | | | | | | |
| Autoclaving | | | | | | | |
| Initial - | | 46.5 | 36.4 | 1.61 | 6.63 | 6.5 | Milky white |
| After | | | | | | | |
| Autoclaving | | | | | | | |
| 1 | 4° C. | 46.2 | 35.8 | 1.61 | 6.63 | 6.5 | Milky white |
| month | 25° C. | 47.6 | 36.9 | 1.60 | 6.63 | 6.5 | Milky white |
| | 40° C. | 47.3 | 36.4 | 1.62 | 6.63 | 6.5 | Milky white |
| 3 | 4° C. | 48.3 | 36.8 | 1.63 | 6.63 | 6.5 | Milky white |
| months | 25° C. | 48.6 | 37.1 | 1.62 | 6.63 | 6.6 | Milky white |
| | 40° C. | 49.1 | 36.4 | 1.61 | 6.63 | 6.4 | Milky white |
| 6 | 4° C. | 47.9 | 36.3 | 1.60 | 5.69 | 6.5 | Milky white |
| months | 25° C. | 47.3 | 36.1 | 1.60 | 5.69 | 6.5 | Milky white |
| | 40° C. | 48.8 | 34.0 | 1.60 | 5.69 | 6.5 | Milky white |

Activities of posaconazole against a broad spectrum of infectious agents have been tested in vitro. Tables 10 and 11 display a subset of the results of this in vitro testing, showing some of those infectious agents against which posaconazole is most active.

TABLE 10
Geometric Mean MIC and MIC[90] Values (μg/mL) for Strains Tested (n) in Posaconazole (POS),
Fluconazole (FLU) and Itraconazole (ITZ)

| | POS | | | FLZ | | | ITZ | | |
|---|---|---|---|---|---|---|---|---|---|
| Organism | n | Mean | MIC[90] | n | Mean | MIC[90] | n | Mean | MIC[90] |
| Aspergillus flavus | 241 | 0.079 | 0.25 | 94 | 220.898 | 256.0 | 203 | 0.213 | 1.0 |
| Aspergillus fumigatus | 2,158 | 0.118 | 0.5 | 735 | 247.922 | 512.0 | 1560 | 0.397 | 1.0 |
| Aspergillus nidulans | 33 | 0.055 | 0.25 | 8 | 76.109 | (32.0-128.0) | 21 | 0.186 | 0.5 |
| Aspergillus niger | 171 | 0.195 | 0.5 | 64 | 234.753 | 256.0 | 153 | 0.834 | 2.0 |
| Aspergillus sydowii | 8 | 0.177 | (0.031-0.5) | 7 | 115.933 | (64.0-256.0) | 8 | 0.500 | (0.125-2.0) |
| Aspergillus terreus | 100 | 0.052 | 0.25 | 37 | 208.327 | 256.0 | 56 | 0.229 | 0.5 |
| Aspergillus ustus | 7 | 1.641 | (0.25-8.0) | 7 | 172.275 | (64.0-256.0) | 7 | 0.906 | (0.125-2.0) |
| Candida albicans | 8,847 | 0.037 | 0.25 | 7,879 | 0.415 | 2.0 | 7,686 | 0.064 | 0.25 |
| Candida dubliniensis | 339 | 0.062 | 0.25 | 231 | 0.454 | 32.0 | 197 | 0.107 | 0.5 |
| Candida glabrata | 2,507 | 0.672 | 2.0 | 2,197 | 9.719 | 64.0 | 2,188 | 0.853 | 4.0 |
| Candida krusei | 496 | 0.335 | 1.0 | 386 | 32.521 | 64.0 | 383 | 0.576 | 1.0 |
| Candida parapsilosis | 2,126 | 0.073 | 0.125 | 1,916 | 0.910 | 2.0 | 1,903 | 0.161 | 0.5 |
| Cryptococcus laurentii | 5 | 0.095 | (0.008-0.5) | 3 | 5.040 | (4.0-8.0) | 3 | 0.397 | (0.25-0.5) |
| Cryptococcus neoformans | 1,427 | 0.119 | 0.25 | 1,237 | 1.781 | 8.0 | 1,269 | 0.444 | 4.0 |
| Coccidioides immitis | 50 | 0.304 | 1.0 | 25 | 16.450 | 32.0 | 50 | 0.198 | 0.25 |
| Fonsecae pedrosoi | 4 | 0.250 | (0.25) | 2 | 64.000 | (64.0) | 4 | 0.063 | (0.008-0.5) |
| Histoplasma capsulatum | 58 | 0.038 | 0.25 | 8 | 19.027 | (8.0-32.0) | 53 | 0.018 | 0.063 |
| Pseudallescheria boydii | 66 | 0.365 | 1.0 | 41 | 41.237 | 128.0 | 61 | 0.506 | 1.0 |
| Alternaria spp | 13 | 0.101 | 0.25 | 0 | — | — | 13 | 0.326 | 1.0 |
| Exophiala dermatidis | 3 | 0.125 | (0.125) | 2 | 8.000 | (8.0) | 2 | 1.000 | (1.0) |
| Exophiala jeanselmei | 10 | 0.287 | 0.5 | 0 | — | — | 10 | 0.467 | 1.0 |
| Exophiala moniliae | 2 | 0.016 | (0.016) | 0 | — | — | 2 | 0.031 | (0.031) |
| Fusarium spp | 38 | 2.319 | 16.0 | 27 | 249.512 | 256.0 | 30 | 13.300 | 16.0 |
| Ramichloridium obovoideum | 2 | 0.044 | (0.031-0.063) | 2 | 22.627 | (16.0-32.0) | 2 | 0.016 | (0.016) |
| Rhizomucor spp | 2 | 0.016 | (0.016) | 0 | — | — | 2 | 0.016 | (0.016) |
| Mucor spp | 17 | 0.694 | 16.0 | 10 | 207.937 | 256.0 | 12 | 2.378 | 16.0 |
| Rhizopus spp | 29 | 1.000 | 4.0 | 19 | 229.461 | 256.0 | 21 | 3.281 | 16.0 |
| Candida famata | 44 | 0.125 | 0.5 | 44 | 4.084 | 32.0 | 27 | 0.348 | 1.0 |
| Candida guilliermondii | 143 | 0.178 | 0.5 | 106 | 4.000 | 32.0 | 82 | 0.479 | 1.0 |
| Candida lusitaniae | 306 | 0.048 | 0.125 | 221 | 0.627 | 2.0 | 202 | 0.216 | 1.0 |
| Candida kefyr | 53 | 0.081 | 0.25 | 51 | 0.500 | 4.0 | 39 | 0.188 | 0.5 |
| Candida rugosa | 26 | 0.039 | 0.5 | 21 | 3.391 | 16.0 | 17 | 0.196 | 4.0 |
| Candida tropicalis | 1,645 | 0.081 | 0.25 | 1,476 | 0.961 | 4.0 | 1,450 | 0.167 | 0.5 |
| Candida zeylanoides | 4 | 0.031 | (0.008-0.25) | 4 | 0.354 | (0.125-1.0) | 4 | 0.105 | (0.031-0.5) |
| Kluyveromyces marxianus | 6 | 0.079 | (0.063-0.25) | 6 | 0.500 | (0.25-1.0) | 6 | 0.070 | (0.031-0.125) |
| Saccharomyces cerevisiae | 86 | 0.249 | 1.0 | 59 | 2.845 | 16.0 | 54 | 0.418 | 2.0 |
| Yarrowia lipolytica | 5 | 0.144 | (0.016-1.0) | 5 | 1.741 | (0.125-32.0) | 0 | — | — |
| Pichia anomala | 13 | 0.689 | 1.0 | 12 | 2.670 | 4.0 | 12 | 0.375 | 1.0 |
| Pichia etchel | 2 | 0.125 | (0.125) | 2 | 0.125 | (0.125) | 0 | — | — |
| Pichia ohmeri | 1 | 0.016 | (0.016) | 1 | 4.000 | (4.0) | 0 | — | — |
| Trichosporon spp | 6 | 0.630 | (0.5-1.0) | 6 | 12.699 | (4.0-64.0) | 6 | 1.123 | (0.5-2.0) |
| Bjerkandera adusta | 14 | 0.250 | 0.25 | 14 | 4.000 | 4.0 | 14 | 0.057 | 0.063 |
| Blastomyces dermatitidis | 43 | 0.053 | 0.125 | 38 | 2.191 | 16.0 | 38 | 0.045 | 2.0 |
| Epidermophyton floccosum | 70 | 0.029 | 0.125 | 15 | 1.447 | 2.0 | 18 | 0.088 | 32.0 |
| Paracoccidioides brasiliensis | 13 | 0.048 | 0.125 | 13 | 0.766 | 4.0 | 13 | 0.025 | 0.063 |
| Scedosporium apiospermum | 32 | 0.173 | 1.0 | 15 | 84.449 | 256.0 | 26 | 1.341 | 32.0 |
| Sporothrix schenckii | 16 | 0.771 | 2.0 | 0 | — | — | 11 | 0.302 | 0.5 |
| Wangiella dermatitidis | 4 | 0.088 | (0.063-0.125) | 2 | 256.000 | (256.0) | 4 | 0.500 | (0.063-1.0) |
| Absidia spp | 8 | 0.177 | (0.031-0.5) | 6 | 143.675 | (128.0-256.0) | 8 | 0.229 | (0.063-2.0) |
| Apophysomyces spp | 9 | 0.340 | (0.031-4.0) | 4 | 128.000 | (128.0) | 6 | 0.707 | (0.031-8.0) |
| Bipolaris spp | 8 | 0.354 | (0.125-1.0) | 2 | 64.000 | (64.0) | 2 | 0.250 | (0.25) |
| Curvularia spp | 5 | 0.072 | (0.031-0.125) | 2 | 16.000 | (16.0) | 2 | 0.500 | (0.5) |
| Microsporum audouinii | 1 | 0.250 | (0.25) | 0 | — | — | 1 | 0.125 | (0.125) |
| Microsporum canis | 86 | 0.034 | 0.5 | 11 | 2.000 | 4.0 | 23 | 0.041 | 2.0 |
| Microsporum fulvum | 1 | 0.500 | (0.5) | 0 | — | — | 1 | 4.000 | (4.0) |
| Microsporum gypseum | 5 | 0.042 | (0.008-0.5) | 4 | 16.000 | (4.0-128.0) | 4 | 0.044 | (0.016-0.5) |
| Microsporum persicolor | 1 | 0.250 | (0.25) | 1 | 128.000 | (128.0) | 1 | 0.500 | (0.5) |
| Paecilomyces spp | 16 | 0.239 | 0.5 | 14 | 141.323 | 256.0 | 14 | 0.640 | 2.0 |
| Penicillium spp | 93 | 0.308 | 1.0 | 66 | 220.996 | 256.0 | 83 | 0.853 | 2.0 |
| Trichophyton mentagrophytes | 84 | 0.036 | 0.125 | 30 | 9.190 | 64.0 | 29 | 0.041 | 0.25 |
| Trichophyton raubitschekii | 1 | 0.250 | (0.25) | 1 | 128.000 | (128.0) | 1 | 0.250 | (0.25) |
| Trichophyton rubrum | 148 | 0.047 | 0.25 | 93 | 4.407 | 32.0 | 91 | 0.074 | 0.25 |
| Trichophyton soudanense | 1 | 0.500 | (0.5) | 1 | 128.000 | (128.0) | 1 | 4.000 | (4.0) |
| Trichophyton spp | 10 | 0.036 | 0.064 | 0 | — | — | 0 | — | — |
| Trichophyton terrestre | 1 | 0.125 | (0.125) | 1 | 1.000 | (1.0) | 1 | 0.250 | (0.25) |
| Trichophyton tonsurans | 74 | 0.029 | 0.125 | 24 | 3.775 | 32.0 | 23 | 0.036 | 0.063 |

TABLE 11
Geometric Mean MIC and MIC[90] Values (μg/mL) for # Strains Tested (n) in Posaconazole (POS),
Amphotericin (AMB) and Voriconazole (VOR)

| | POS | | | AMB | | | VOR | | |
|---|---|---|---|---|---|---|---|---|---|
| Organism | n | Mean | MIC[90] | n | Mean | MIC[90] | n | Mean | MIC[90] |
| Aspergillus flavus | 241 | 0.079 | 0.25 | 177 | 0.910 | 2.0 | 89 | 0.339 | 1.0 |
| Aspergillus fumigatus | 2.158 | 0.118 | 0.5 | 1.567 | 0.683 | 1.0 | 1149 | 0.282 | 0.5 |
| Aspergillus nidulans | 33 | 0.055 | 0.25 | 20 | 0.758 | 2.0 | 6 | 0.070 | (0.031-0.125) |
| Aspergillus niger | 171 | 0.195 | 0.5 | 152 | 0.360 | 1.0 | 101 | 0.480 | 2.0 |
| Aspergillus sydowii | 8 | 0.177 | (0.031-0.5) | 3 | 1.260 | (1.0-2.0) | 3 | 0.397 | (0.25-1.0) |
| Aspergillus terreus | 100 | 0.052 | 0.25 | 54 | 1.759 | 4.0 | 22 | 0.312 | 0.5 |
| Aspergillus ustus | 7 | 1.641 | (0.25-8.0) | 7 | 0.673 | (0.25-1.0) | 4 | 1.189 | (0.25-2.0) |
| Candida albicans | 8.847 | 0.037 | 0.25 | 6.651 | 0.686 | 1.0 | 3.790 | 0.021 | 0.063 |
| Candida dubliniensis | 339 | 0.062 | 0.25 | 211 | 0.513 | 1.0 | 177 | 0.028 | 0.125 |
| Candida glabrata | 2.507 | 0.672 | 2.0 | 1.881 | 0.798 | 1.0 | 1.264 | 0.305 | 2.0 |
| Candida krusei | 496 | 0.335 | 1.0 | 282 | 0.976 | 2.0 | 210 | 0.346 | 0.5 |
| Candida parapsilosis | 2.126 | 0.073 | 0.125 | 1.655 | 0.761 | 1.0 | 1.011 | 0.036 | 0.125 |
| Cryptococcus laurentii | 5 | 0.095 | (0.008-0.5) | 3 | 0.794 | (0.5-1.0) | 1 | 0.250 | (0.25) |
| Cryptococcus neoformans | 1.427 | 0.119 | 0.25 | 1.122 | 0.667 | 1.0 | 277 | 0.054 | 0.125 |
| Coccidioides immitis | 50 | 0.304 | 1.0 | 25 | 0.390 | 0.5 | 0 | — | — |
| Fonsecae pedrosoi | 4 | 0.250 | (0.25) | 2 | 1.000 | (1.0) | 1 | 0.500 | (0.5) |
| Histoplasma capsulatum | 58 | 0.038 | 0.25 | 53 | 0.250 | 0.5 | 0 | — | — |
| Pseudallescheria boydii | 66 | 0.365 | 1.0 | 41 | 1.718 | 4.0 | 3 | 0.250 | (0.125-0.5) |
| Alternaria spp | 13 | 0.101 | 0.25 | 13 | 0.852 | 4.0 | 0 | — | — |
| Exophiala dermatidis | 3 | 0.125 | (0.125) | 2 | 0.500 | (0.5) | 1 | 0.063 | (0.063) |
| Exophiala jeanselmei | 10 | 0.287 | 0.5 | 10 | 0.660 | 1.0 | 9 | 0.794 | (0.5-1.0) |
| Exophiala moniliae | 2 | 0.016 | (0.016) | 2 | 0.177 | (0.125-0.25) | 0 | — | — |
| Fusarium spp | 38 | 2.319 | 16.0 | 30 | 1.203 | 2.0 | 14 | 4.416 | 16.0 |
| Ramichloridium obovoideum | 2 | 0.044 | (0.031-0.063) | 2 | 1.000 | (1.0) | 0 | — | — |
| Rhizomucor spp | 2 | 0.016 | (0.016) | 2 | 0.063 | (0.063) | 1 | 2.000 | (2.0) |
| Mucor spp | 17 | 0.694 | 16.0 | 15 | 0.274 | 1.0 | 8 | 24.675 | (1.0-128.0) |
| Rhizopus spp | 29 | 1.000 | 4.0 | 29 | 0.635 | 2.0 | 9 | 8.000 | (1.0-32.0) |
| Candida famata | 44 | 0.125 | 0.5 | 28 | 0.841 | 2.0 | 5 | 0.072 | (0.008-0.5) |
| Candida guilliermondii | 143 | 0.178 | 0.5 | 76 | 0.553 | 1.0 | 26 | 0.112 | 8.0 |
| Candida lusitaniae | 306 | 0.048 | 0.125 | 164 | 0.506 | 1.0 | 89 | 0.023 | 0.063 |
| Candida kefyr | 53 | 0.081 | 0.25 | 25 | 0.779 | 1.0 | 14 | 0.031 | 0.031 |
| Candida rugosa | 26 | 0.039 | 0.5 | 17 | 0.665 | 1.0 | 15 | 0.072 | 0.5 |
| Candida tropicalis | 1.645 | 0.081 | 0.25 | 1.209 | 0.774 | 1.0 | 765 | 0.075 | 0.5 |
| Candida zeylanoides | 4 | 0.031 | (0.008-0.25) | 3 | 1.000 | (1.0) | 3 | 0.039 | (0.008-0.125) |
| Kluyveromyces marxianus | 6 | 0.079 | (0.063-0.25) | 6 | 1.000 | (1.0) | 0 | — | — |
| Saccharomyces cerevisiae | 86 | 0.249 | 1.0 | 38 | 0.775 | 1.0 | 22 | 0.050 | 0.125 |
| Yarrowia lipolytica | 5 | 0.144 | (0.016-1.0) | 2 | 0.500 | (0.5) | 0 | — | — |
| Pichia anomala | 13 | 0.689 | 1.0 | 10 | 0.707 | 1.0 | 0 | — | — |
| Pichia etchel | 2 | 0.125 | (0.125) | 2 | 0.063 | (0.063) | 0 | — | — |
| Pichia ohmeri | 1 | 0.016 | (0.016) | 0 | — | — | 0 | — | — |
| Trichosporon spp | 6 | 0.630 | (0.5-1.0) | 5 | 1.320 | (1.0-2.0) | 1 | 0.125 | (0.125) |
| Bjerkandera adusta | 14 | 0.250 | 0.25 | 14 | 0.215 | 0.25 | 14 | 0.216 | 0.25 |
| Blastomyces dermatitidis | 43 | 0.053 | 0.125 | 38 | 0.153 | 0.5 | 0 | — | — |
| Epidermophyton floccosum | 70 | 0.029 | 0.125 | 0 | — | — | 10 | 0.015 | 0.016 |
| Paracoccidioides brasiliensis | 13 | 0.048 | 0.125 | 13 | 0.096 | 0.25 | 0 | — | — |
| Scedosporium apiospermum | 32 | 0.173 | 1.0 | 27 | 2.274 | 8.0 | 14 | 0.098 | 0.5 |
| Sporothrix schenckii | 16 | 0.771 | 2.0 | 10 | 0.574 | 1.0 | 0 | — | — |
| Wangiella dermatitidis | 4 | 0.088 | (0.063-0.125) | 3 | 0.630 | (0.25-1.0) | 1 | 1.000 | (1.0) |
| Absidia spp | 8 | 0.177 | (0.031-0.5) | 8 | 0.545 | (0.25-2.0) | 6 | 40.318 | (8.0-128.0) |
| Apophysomyces spp | 9 | 0.340 | (0.031-4.0) | 7 | 0.500 | (0.031-4.0) | 5 | 42.224 | (16.0-128.0) |
| Bipolaris spp | 8 | 0.354 | (0.125-1.0) | 2 | 0.250 | (0.25) | 1 | 1.000 | (1.0) |
| Curvularia spp | 5 | 0.072 | (0.031-0.125) | 2 | 0.500 | (0.5) | 1 | 0.125 | (0.125) |
| Microsporum audouinii | 1 | 0.250 | (0.25) | 0 | — | — | 0 | — | — |
| Microsporum canis | 86 | 0.034 | 0.5 | 0 | — | — | 10 | 0.018 | 0.031 |
| Microsporum fulvum | 1 | 0.500 | (0.5) | 0 | — | — | 0 | — | — |
| Microsporum gypseum | 5 | 0.042 | (0.008-0.5) | 0 | — | — | 0 | — | — |
| Microsporum persicolor | 1 | 0.250 | (0.25) | 0 | — | — | 0 | — | — |
| Paecliomyces spp | 16 | 0.239 | 0.5 | 14 | 1.104 | 16.0 | 8 | 0.324 | (0.031-2.0) |
| Penicillium spp | 93 | 0.308 | 1.0 | 83 | 1.025 | 4.0 | 35 | 0.622 | 2.0 |
| Trichophyton mentagrophytes | 84 | 0.036 | 0.125 | 2 | 1.000 | (1.0) | 11 | 0.038 | 0.25 |
| Trichophyton raubitschekii | 1 | 0.250 | (0.25) | 0 | — | — | 0 | — | — |
| Trichophyton rubrum | 148 | 0.047 | 0.25 | 0 | — | — | 10 | 0.021 | 0.063 |
| Trichophyton soudanense | 1 | 0.500 | (0.5) | 0 | — | — | 0 | — | — |
| Trichophyton spp | 10 | 0.036 | 0.064 | 0 | — | — | 0 | — | — |
| Trichophyton terrestre | 1 | 0.125 | (0.125) | 0 | — | — | 0 | — | — |
| Trichophyton tonsurans | 74 | 0.029 | 0.125 | 3 | 1.260 | (0.5-8.0) | 11 | 0.043 | 0.063 |

Pharmacokinetics

The pharmacokinetic characteristics of the posaconazole formulation were evaluated in a Phase-1, single-site, randomized, evaluator-blinded (within dose level), placebo-controlled, rising-single-dose study, with up to six groups of 12 healthy subjects. The purpose of the study was to evaluate the safety, tolerability, and pharmacokinetics of the posaconazole intravenous drug product formulation (hereinafter referred to as “POS IV”) when delivered intravenously. Table 12 shows the POS IV formulation, and Table 13 shows the physical characteristics of this formulation after sterilization, but before dilution in 5% dextrose.

TABLE 12
POS IV Formulation, 50 mg/mL

| Ingredient | mg/mL |
|---|---|
| Posaconazole, micronized | 50.0 |
| 1-Palmitoyl-2-Oleoyl-sn-glycero-3-Phosphocholine, | 40.0 |
| Powder, Endotoxin tested (POPC) | |
| Sodium Phoshate, Monobasic, Monohydrate, Crystal, USP | 0.040 |
| Sodium Phosphate, Dibasic, Anhydrous, USP | 1.378 |
| Trehalose | 250.0 |
| Sodium Hydroxide, N.F. (1.0 N) | AN |
| Phosphoric Acid, N.F. (20% w/w) | AN |
| Water Injection, USP, q.s. ad | 1 |

AN = As needed for pH adjustment

TABLE 13
Physical Characteristics of POS IV, 50 mg/mL

| Description: | milky liquid |
|---|---|
| pH: | ˜6-8 |

| | |
|---|---|
| Osmolality: | ˜875 mOsm/kg (isotonic upon |
| | admixing) |
| Particulates: | meets USP Particulate Matter tests |
| Particle Size: | Median 1.0-1.8 μm 100% <˜10 μm |
| Sterility/ | meets USP tests |
| Endotoxins: | |

Within each dose group, subjects were randomized on Day 1 according to a computer-generated schedule provided by Schering-Plough Research Institute.

Healthy adult males or females 18 to 45 years of age having body mass indices (BMIs) of 19 to 27 were eligible for inclusion in Groups 1 to 4 of the study. Healthy adult males or females 18 to 45 years of age having BMIs of 19 to 27 and having body weights of >60 kg were eligible for inclusion in Groups 5 and 6 of the study.

POS IV (50 mg/mL) was diluted in 5% dextrose in water (D₅W) in IV bags. Subjects assigned to active drug received in a 100-mL volume one of the following single doses administered intravenously over 1 hour: Group 1, 25 mg; Group 2, 50 mg; Group 3, 100 mg; Group 4, 200 mg; Group 5, 400 mg; Group 6, a 125-mL volume a single dose of 600 mg administered intravenously over 1 hour and 15 minutes.

Blood samples (10 mL each) for the determination of posaconazole concentrations were collected immediately prior to dosing (0 hour), and at 0.25, 0.5, 1, 1.25, 1.5, 2, 3, 4, 6, 8, 10, 12, 14, 16, 24, 48, 72, 96, and 120 hours after the start of infusion, as well as on the follow-up visit on Day 14. The blood samples were collected into two tubes containing ethylenediaminetetraacetate salt (EDTA) with each tube containing 4 mL to 5 mL of blood, one tube for determination of posaconazole in whole blood and other in plasma. For determination of posaconazole in plasma, the tube of blood (4 mL to 5 mL) was centrifuged within approximately 15 minutes of collection at approximately 4° C. and 1500 g for 10 minutes to completely separate red blood cells from plasma. All blood and plasma samples were immediately frozen to at least −20° C. and maintained in the frozen state until assayed. The blood and plasma concentrations of posaconazole were determined using validated high performance liquid chromatographic-mass spectrometric (LC-MS/MS) assays. The lower limit of quantitation (LLOQ) of this assay was 5.0 ng/mL and the calbration range was 5 to 5000 ng/niL.

The following pharmacokinetic parameters were determined: maximum plasma concentration (Cₘₐₓ); time of maximum plasma concentration (Tₘₐₓ); the area under the plasma concentration versus time curve to infinity (AUC[I]); the area under the plasma concentration versus time curve to the final measurable sampling time (AUC[tf]); terminal phase half-life (t_{1/2}); total body clearance (CL); and, volume of distribution at steady-state (Vdss).

Posaconazole blood and plasma concentrations above the LLOQ were used for the non-compartmental pharmacokinetic analyses. Pharsight® Knowledgebase Server®: version 2.0.1 (PKS) with WinNonlin version 4.0.1 (Pharsight Corporation, Cary, N.C.) was used to conduct the pharmacokinetic analysis. The Cₘₐₓ and Tₘₐₓ were the observed values. The terminal phase rate constant (k) was calculated as the negative of the slope of the log-linear terminal portion of the serum concentration-time curve using linear regression. The terminal phase half-life, t_{1/2}, was calculated as 0.693/k.

The area under the serum concentration-time curve from time 0 to the time of final quantifiable sample [AUC(tf)] was calculated using the linear trapezoidal rule. AUC(tf) was then extrapolated to infinity (I) as follows:
AUC(I)=AUC(tf)+Ces(tf)/k
where Ces(tf) is the estimated concentration determined from linear regression at final measurable sampling time, tf.

Total body clearance, CL, was calculated by the following equation:
CL=Dose/AUC(I)

The apparent volume of distribution at steady-state, Vdss, was calculated as:
Vdss=CL×MRT
where MRT is the mean residence time (adjusted for infusion duration) determined from moment analysis.

The observed single dose plasma concentrations were used for pharmacokinetic modeling and simulation and to project steady-state concentrations for once-a-day (QD) dosing regimen. A nonparameteric superposition method was used for the pharmacokinetic modeling and simulation under the assumption of linear pharmacokinetics (see Gibaldi M, Perrier D., Pharmacokinetics, 2nd ed., New York: Marcel Dekker, Inc., 1982:409-17).

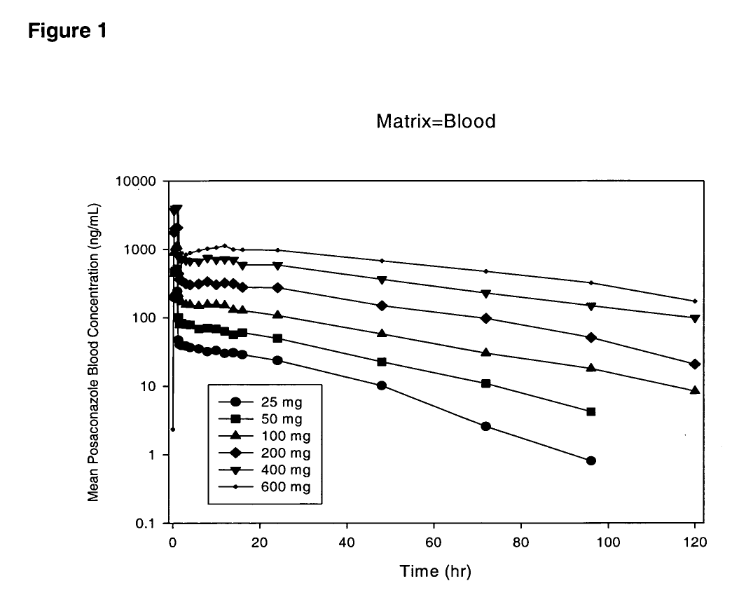

After cessation of infusion of POS IV, posaconazole plasma concentrations declined unusually rapidly, and then, surprisingly, increased subsequently, followed by a slow declining terminal phase (see FIGS. 1-4). This pharmacokinetic profile is believed to be atypical and unique among known azoles. Moreover, this pharmacokinetic pattern was also observed after the intravenous administration of posaconazole in animals. It is indicative of a rapid distribution of posaconazole to the liver and spleen and subsequent slow release from these tissues. Therefore, as noted in the literature with respect to another pharmaceutically active agent (Townsend R W, Zutshi A, Bekersky I., “Biodistribution of 4-[14C]cholesterol-Ambisome following a single intravenous administration to rats”, Drug Metabolism and Disposition. 2001;29:681-5), POS IV may be initially sequestrated in tissues, such as the liver and spleen, via uptake through the reticuloendothelial system (“RES”). Although not intended to be limited to any single mechanism of action, it is believed that the resulting high concentrations of posaconazole in these tissues due to sequestration of the drug may contribute to enhanced anti-infective activity, since these tissues are often the sites of infection.

In order to determine the target dosing for intravenous administration, it was necessary to determine a target range for mean C_{avg} and mean Cₘₐₓ. Previous studies on orally administered posaconazole are instructive in this regard. Table 14 displays pharmacokinetic data resulting from such oral administration, arranged by quartile based on the observed range of posaconazole plasma concentration values. For each quartile, the response rate for apergfilosis is displayed.

TABLE 14
Pharmacokinetic Results of Orally Administered
Posaconazole

| | Plasma Cₘₐₓ | | | | |
|---|---|---|---|---|---|
| | (ng/mL) | | Plasma C_{avg} (ng/mL)(b) | | Response |
| Quartile | Mean(a) | % CV | Mean(a) | % CV | (%) |
| 1 | 142 | 51 | 134 | 45 | 24 |
| 2 | 467 | 27 | 411 | 21 | 53 |
| 3 | 852 | 15 | 719 | 12 | 53 |
| 4 | 1480 | 16 | 1250 | 28 | 71 |

a: n = 17, with the exception of Quartile 4 where n = 16
b: C_{avg} = average plasma concentration across time points in the same subject

The table shows that the target mean Cₘₐₓ for a response rate of at least 50% should be in the range of 467 to 1480 ng/mL, or higher. The pharmacokinetic modeling and steady-state projection based on the pharmacokinetic results of POS IV once-a-day (QD) dosing regimen show that the projected posaconazole mean Cₘₐₓ at a 100 mg POS IV QD dose will be 714 ng/mL (see Table 17, 100 mg dose), which exceeds 467 ng/mL, the minimum clinically relevant mean plasma Cₘₐₓ. The data in Table 17 suggest that there exists a dose between 50 and 100 mg which will result in the minimum clinically relevant mean plasma Cₘₐₓ of about 467. However, in terminal disease states, it is desirable to treat the patient with the maximum tolerated dose. Thus, having established that a dose of 100 mg is projected to achieve the minimum clinically relevant mean plasma Cₘₐₓ, it may be desirable to dose at higher quantities, e.g., 200 mg, 400 mg, or 600 mg, subject to tolerability.

After intravenous administration of POS IV formulation, posaconazole was slowly eliminated from plasma with an average terminal half-life of 21 to 39 hours. The half-life was higher at the higher dose compared to that at lower dose groups (see Tables 15 and 16), in a range of about 15 hours (with a 100 mg dose) to about 51 hours (with a 400 mg dose). A long half-life is desirable as it provides the sustained and high plasma concentration of antifungal agent over the entire dosing interval, likely contributing to better antifungal activity. The systemic clearance appeared to decrease with increasing doses and ranged from 13 to 6 L/hr (see Tables 15 and 16). The mean volume of distribution was large (326 to 408 L) exceeding total body water volume of about 40 L. This suggests extensive tissue distribution and penetration into the tissues, a characteristic that likely contributes to enhanced anti-infective activity. The range in the data for Vdss was from 219 to 516 L. This is consistent with the coefficient of variation of the data, which suggests that the volume distribution could have a range of 200 to 500 L.

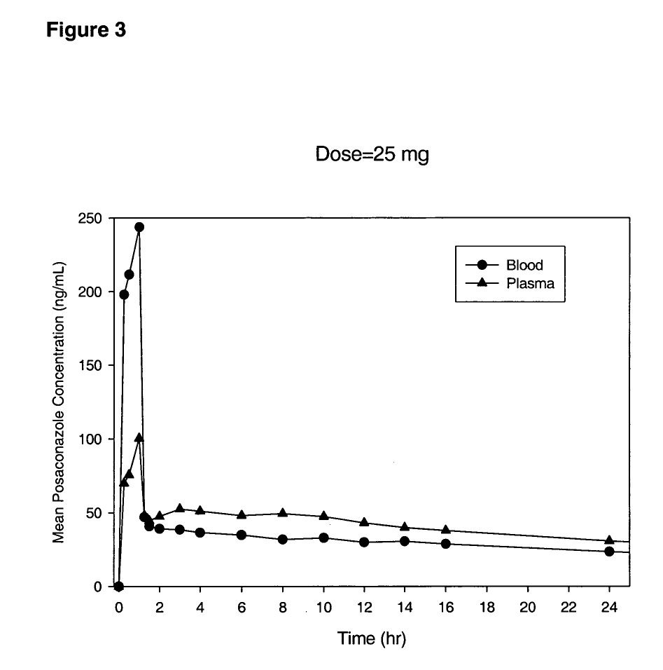

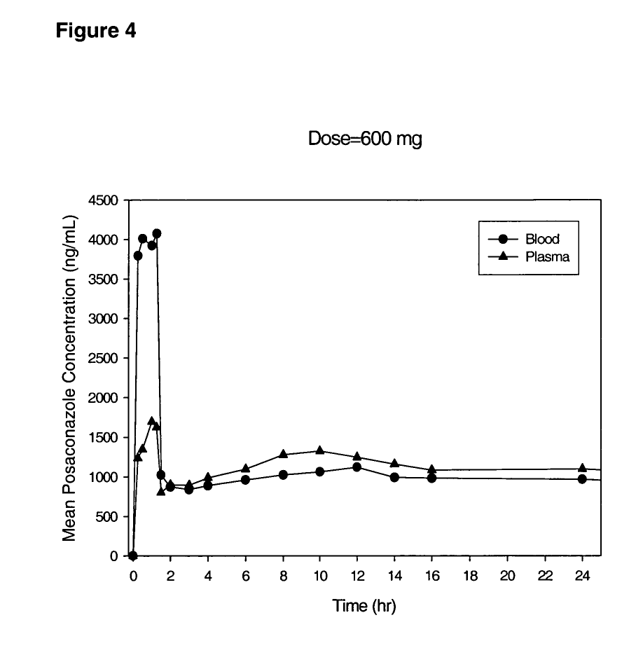

The preferable ratios of blood to plasma posaconazole Cₘₐₓ and AUC values are shown in Tables 18 and 19. Overall posaconazole exposure (AUC) was higher in plasma compared to that in blood (see Tables 18 and 19—AUC ratio). However, the posaconazole concentrations were greater in blood than in plasma during the infusion and approximately up to 1 hr post-infusion (see FIGS. 3 and 4; Tables 18 and 19, Cₘₐₓ ratio). These unique differences between blood and plasma concentrations may contribute to the preferential sequestration of posaconazole in the liver and spleen, as previously noted. The coefficient of variation of the data suggests that the ratio of blood to plasma posaconazole Cₘₐₓ could have a range of 1.8 to 3.5 for single dose infused over 1 hour to deliver 25-600 mg of posaconazole. The coefficient of variation of the data suggests that the ratio of blood to plasma posaconazole Cₘₐₓ could have a range of 1.0 to 2.3 at steady state when posaconazole is infused over about 1 hour, and repeated on a 24-hour basis, to deliver 25-600 mg of posaconazole. A ratio different than that shown in Table 18 may provide different distribution properties that could translate into differences in anti-infective activity.

TABLE 15
Mean (n = 9) Posaconazole Blood Pharmacokinetic Parameters in Subjects after a
Single-dose 1 hr IV Infusion of Posaconazole IV Formulation

| | 25 mg | | 50 mg | | 100 mg | | 200 mg | | 400 mg | | 600 mg | |
|---|---|---|---|---|---|---|---|---|---|---|---|---|
| Parameter | Mean | CV(%) | Mean | CV(%) | Mean | CV(%) | Mean | CV(%) | Mean | CV(%) | Mean | CV(%) |
| Cmax | 244 | 18 | 540 | 11 | 1130 | 22 | 2150 | 8 | 4330 | 14 | 4410 | 9 |
| (ng/mL) | | | | | | | | | | | | |
| Tmax^{a} (hr) | 1 | — | 1 | — | 1 | — | 1 | — | 1 | — | 1.25 | — |
| range | 0.5-1.0 | | 0.5-1.0 | | 0.5-1.0 | | 0.5-1.0 | | 0.25-1.0 | | 0.5-1.25 | |
| AUC(tf) | 1450 | 28 | 3370 | 16 | 8110 | 21 | 19500 | 20 | 44800 | 23 | 76300 | 15 |
| (ng · hr/mL) | | | | | | | | | | | | |
| AUC(l) | 1680 | 27 | 3620 | 17 | 8630 | 22 | 20700 | 21 | 50900 | 29 | 87300 | 16 |
| (ng · hr/mL) | | | | | | | | | | | | |
| t½ (hr) | 21.1 | 25 | 22.6 | 19 | 27.8 | 23 | 26.5 | 19 | 37.5 | 31 | 39.0 | 22 |
| CL | 15.7 | 23 | 14.2 | 18 | 12.3 | 31 | 10.1 | 24 | 8.33 | 23 | 7.01 | 14 |
| (L/hr) | | | | | | | | | | | | |
| Vdss (L) | 408 | 14 | 395 | 11 | 408 | 12 | 379 | 19 | 427 | 22 | 393 | 24 |

^{a}median

TABLE 16
Mean (n = 9) Posaconazole Plasma Pharmacokinetic Parameters in Subjects
after a Single-dose 1 hr IV Infusion of Posaconazole IV Formulation

| | 25 mg | | 50 mg | | 100 mg | | 200 mg | | 400 mg | | 600 mg | |
|---|---|---|---|---|---|---|---|---|---|---|---|---|
| Parameter | Mean | CV (%) | Mean | CV (%) | Mean | CV (%) | Mean | CV (%) | Mean | CV (%) | Mean | CV (%) |
| Cmax | 103 | 25 | 206 | 26 | 426 | 25 | 898 | 27 | 1780 | 21 | 1850 | 21 |
| (ng/mL) | | | | | | | | | | | | |
| Tmax^{a} (hr) | 1 | — | 1 | — | 1 | — | 1 | — | 1 | — | 1.25 | — |
| range | 0.5-1.0 | | 0.25-4.0 | | 0.5-1.0 | | 0.25-1.0 | | 0.5-1.0 | | 1.0-10.0 | |
| AUC (tf) | 1820 | 35 | 4490 | 18 | 9320 | 22 | 23300 | 21 | 49000 | 29 | 83700 | 16 |
| (ng.hr/mL) | | | | | | | | | | | | |
| AUC (l) | 2040 | 34 | 4740 | 18 | 9890 | 24 | 24700 | 23 | 55400 | 36 | 96700 | 18 |
| (ng.hr/mL) | | | | | | | | | | | | |
| t½ (hr) | 21.4 | 18 | 21.7 | 23 | 26.5 | 25 | 27.1 | 22 | 35.4 | 29 | 39.4 | 23 |
| CL (L/hr) | 13.3 | 29 | 10.9 | 21 | 10.9 | 37 | 8.57 | 27 | 7.84 | 27 | 6.36 | 16 |
| Vdss (L) | 396 | 20 | 331 | 12 | 389 | 13 | 324 | 18 | 378 | 22 | 356 | 23 |

^{a}median

TABLE 17
Mean Projected Steady-state Plasma and Blood Posaconazole PK
Parameters in Subjects Receiving a Daily Dose of POS IV
infusion over 1-1.25 hr

| | Plasma | | | | Blood | | | |
|---|---|---|---|---|---|---|---|---|
| | Cmax | | AUC (0-24 hr) | | Cmax | | AUC (0-24 hr) | |
| | (ng/mL) | | (ng.hr/mL) | | (ng/mL) | | (ng.hr/mL) | |
| Dose | | CV, | | CV, | | CV, | | CV, |
| (mg) | Mean | % | Mean | % | Mean | % | Mean | % |
| 25 | 155 | 26 | 1960 | 36 | 281 | 18 | 1580 | 29 |
| 50 | 335 | 16 | 4690 | 19 | 628 | 10 | 3560 | 17 |
| 100 | 714 | 23 | 9840 | 24 | 1360 | 21 | 8570 | 22 |
| 200 | 1670 | 22 | 24600 | 23 | 2760 | 9 | 20600 | 21 |
| 400 | 3540 | 27 | 54500 | 35 | 5870 | 12 | 50000 | 28 |
| 600 | 5100 | 16 | 94900 | 17 | 7260 | 10 | 85800 | 16 |

TABLE 18
Ratio of Mean Blood and Plasma Posaconazole PK Parameters in
Subjects receiving a Single Dose of POS IV infusion over 1-1.25 hr

| | Blood/Plasma Ratio | | | |
|---|---|---|---|---|
| | Cₘₐₓ | | AUC (I) | |
| Dose | | | | |
| (mg) | Mean | CV, % | Mean | CV, % |
| 25 | 2.42 | 17 | 0.839 | 7 |
| 50 | 2.76 | 25 | 0.772 | 13 |
| 100 | 2.71 | 13 | 0.878 | 6 |
| 200 | 2.50 | 18 | 0.844 | 6 |
| 400 | 2.50 | 19 | 0.933 | 8 |
| 600 | 2.45 | 17 | 0.904 | 5 |

TABLE 19
Ratio of Mean Projected Steady-state Blood and Plasma
Posaconazole PK Parameters in Subjects receiving a Daily Dose
of POS IV infusion over 1-1.25 hr

| | Blood/Plasma Ratio | | | |
|---|---|---|---|---|
| | Cmax | | AUC (τ) | |
| Dose | | | | |
| (mg) | Mean | CV, % | Mean | CV, % |
| 25 | 1.85 | 12 | 0.822 | 9 |
| 50 | 1.90 | 15 | 0.766 | 13 |
| 100 | 1.93 | 12 | 0.877 | 6 |
| 200 | 1.70 | 13 | 0.843 | 7 |
| 400 | 1.72 | 19 | 0.932 | 8 |
| 600 | 1.44 | 10 | 0.908 | 5 |

It is to be understood that all formulations that are bioequivalent to those disclosed herein are also within the scope of the present invention.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

Various publications are cited herein, the disclosures of which are inorporated by reference in their entireties.

What is claimed:

1. A formulation comprising a suspension of posaconazole, stabilized by a phospholipid, in a mixture comprising water, a thermoprotectant, and a buffer system.

2. The formulation of claim 1 wherein said water has been removed by lyophilization.

3. The formulation of claim 1 wherein said formulation has been sterilized by autoclaving.

4. The formulation of claim 1 wherein said formulation has been sterilized by irradiation.

5. The formulation of claim 1 wherein said buffer system comprises sodium phosphate.

6. The formulation of claim 1 wherein said buffer system comprises an organic buffer.

7. The formulation of claim 1 wherein said buffer system comprises at least one of histidine, citric acid, glycine, sodium citrate, ammonium sulfate, or acetic acid.

8. The formulation of claim 1 wherein said buffer system maintains a pH of about 3.0 to about 9.0.

9. The formulation of claim 1 wherein said buffer system maintains a pH of about 6.0 to about 8.0.

10. The formulation of claim 1 wherein said buffer system maintains a pH of about 6.4 to about 7.6.

11. The formulation of claim 1 wherein said phospholipid comprises a natural phospholipid.

12. The formulation of claim 1 wherein said phospholipid comprises a synthetic phospholipid.

13. The formulation of claim 1 wherein said phospholipid comprises a natural phospholipid and a synthetic phospholipid.

14. The formulation of claim 1 wherein said phospholipid comprises 1-palmitoyl-2-oleoyl-sn-glycero-3-phosphocholine (POPC).

15. The formulation of claim 1 wherein said thermoprotectant comprises trehalose.

16. The formulation of claim 1 wherein said phospholipid comprises 1-palmitoyl-2-oleoyl-sn-glycero-3-phosphocholine (POPC), said thermoprotectant comprises trehalose, and said buffer system comprises sodium phosphate.

17. The formulation of claim 1 wherein said posaconazole has a particle size distribution whose median value is between about 1.0 and about 8.0 microns, with not more than about 3000 particles of 10 microns or greater size and not more than about 300 particles of 25 microns or greater size.

18. The formulation of claim 1 wherein said posaconazole has a particle size distribution whose median value is between about 1.0 and about 5.0 microns, with not more than about 3000 particles of 10 microns or greater size and not more than about 300 particles of 25 microns or greater size.

19. The formulation of claim 1 wherein said posaconazole has a particle size distribution whose median value is between about 1.2 and about 4.5 microns, with not more than about 3000 particles of 10 microns or greater size and not more than about 300 particles of 25 microns or greater size.

20. The formulation of claim 16 whose ingredients comprise:

| Ingredient | Concentration range |
|---|---|
| Posaconazole | about 50 mg/ml |
| POPC | about 40 mg/ml |
| Sodium Phosphate, | 0.345 mg/ml |
| monobasic, monohydrate, USP | |
| Sodium Phosphate, dibasic, | 1.065 |
| anhydrous, USP | |
| Trehalose | 250 mg/ml |
| Water for Injection, USP q.s. | 1 ml |
| ad | |

21. The formulation of claim 16 whose ingredients comprise:

| Ingredient | Concentration range |
|---|---|
| Posaconazole | about 1 to about 100 mg/ml |
| POPC | about 10 to about 60 mg/ml |
| Sodium Phosphate, | about 0.01 to about 0.6 mg/ml |
| monobasic, monohydrate, USP | |
| Sodium Phosphate, dibasic, | about 0.04 to about 1.5 mg/ml |
| anhydrous, USP | |
| Trehalose | about 10 to about 300 mg/ml |
| Water for Injection, USP q.s. | about 1 ml |
| ad | |

22. The formulation of claim 16 whose ingredients comprise:

| Ingredient | Concentration range |
|---|---|
| Posaconazole | about 40 to about 60 mg/ml |
| POPC | about 20 to about 50 mg/ml |
| Trehalose | about 100 to about 250 mg/ml |
| Water for Injection, USP q.s. | about 1 ml |
| ad | |

23. The formulation of claim 7 whose ingredients comprise:

| Ingredient | Concentration | |
|---|---|---|
| PosaconazoLe | 50 | mg/ml |
| POPC | 40 | mg/ml |
| Histidine | 3 | mg/ml |
| Citric acid monohydrate | 0.24 | mg/ml |
| Trehalose | 250 | mg/ml |
| Water q.s. ad | 1 | ml |

at a pH of about 6.4.

24. The formulation of claim 1 further comprising an antioxidant.

25. The formulation of claim 24, wherein the antioxidant comprises propyl gallate at a concentration of about 0.02 to about 0.005 mg/ml.

26. The formulation of claim 24, wherein the antioxidant comprises butylated hydroxytoluene at a concentration of about 0.1 to about 0.02 mg/ml.

27. The formulation of claim 24, wherein the antioxidant comprises alpha-D-tocopherol at a concentration of about 0.5 to about 0.01 mg/ml.

28. The formulation of claim 24 whose ingredients comprise:

| Ingredient | Concentration | |
|---|---|---|
| Posaconazole | 50 | mg/ml |
| POPC | 40 | mg/ml |
| Histidme | 3 | mg/ml |
| Citric acid monohydrate | 0.24 | mg/ml |
| Propyl gallate | 0.01 | mg/ml |
| Butylated hydroxytoluene | 0.05 | mg/ml |
| Trehalose | 250 | mg/ml |
| Water q.s. ad | 1 | ml |

at a pH of about 6.4.

29. The formulation of claim 24 whose ingredients comprise:

| Ingredient | Concentration | |
|---|---|---|
| Posaconazole | 50 | mg/ml |
| POPC | 40 | mg/ml |
| Histidme | 3 | mg/ml |
| Citric acid monohydrate | 0.24 | mg/ml |
| Alpha-D-tocopherol | 0.05 | mg/ml |
| Trehalose | 250 | mg/ml |
| Water q.s. ad | 1 | ml |

at a pH of about 6.5.

30. The formulation of claim 1 wherein the wt. ratio of phospholipid to posaconazole is between about 60:1 and about 1:10.

31. The formulation of claim 1 wherein the wt. ratio of phospholipid to posaconazole is between about 1:1 and about 1:5.

32. The formulation of claim 1 wherein the wt. ratio of phospholipid to posaconazole is between about 1:1 and about 4:5.

33. The formulation of claim 1 wherein the wt. ratio of thermoprotectant to posaconazole is between about 300:1 and about 1:10.

34. The formulation of claim 1 wherein the wt. ratio of thermoprotectant to posaconazole is between about 1:1 and about 6:1.

35. The formulation of claim 1 wherein the wt. ratio of thermoprotectant to phospholipid is between about 30:1 and about 1:6.

36. The formulation of claim 1 wherein the wt. ratio of thermoprotectant to phospholipid is between about 5:4 and about 30:4.

37. A method of treating or preventing an infection in an animal in need thereof which comprises administering to said animal an effective amount of the formulation of claim 20.

38. The method of claim 37 wherein said infection is caused by a fungus or a parasite.

39. The method of claim 37 wherein said infection is one or more selected from the group consisting of:
oropharyngeal or esophageal candidiasis;
refractory oropharyngeal and esophageal candidiasis;
invasive aspergillosis, candidiasis, fusariosis, scedosporiosis, infections due to dimorphic fungi, zygomycosis, and invasive infections due to rare molds and yeasts;
invasive mycoses in patients who are refractory to, or intolerant of, other therapies;
Candidiasis, invasive mold infections in patients who have undergone intensive chemotherapy and/or radiation therapy for hematologic malignancies, bone marrow or peripheral stem cell transplant conditioning regimens, and patients receiving combination immunosuppressive therapy for the treatment of acute or chronic graft-versus-host disease or prevention of solid organ transplantation; Chagas disease;
and, Leishmaniasis.

40. A method of treating or preventing an infection inan animal in need thereof which comprises administering to said animal an effective amount of the formulation of any of claims 1, 21, 22, 23, 28 or 29.

41. The method of claim 40 wherein said infection is caused by a fungus or a parasite.

42. The method of claim 40 wherein said infection is one or more selected from the group consisting of:
oropharyngeal or esophageal candidiasis;
refractory oropharyngeal and esophageal candidiasis;
invasive aspergillosis, candidiasis, fusariosis, scedosporiosis, infections due to dimorphic fungi, zygomycosis, and invasive infections due to rare molds and yeasts;
invasive mycoses in patients who are refractory to or intolerant of other therapies;
Candidiasis, invasive mold infections in patients who have undergone intensive chemotherapy and/or radiation therapy for hematologic malignancies, bone marrow or peripheral stem cell transplant conditioning regimens, and patients receiving combination immunosuppressive therapy for the treatment of acute or chronic graft-versus-host disease or prevention of solid organ transplantation; Chagas disease; and,
Leishmaniasis.

43. The method of claim 37 wherein said formulation is administered intravenously.

44. The method of claim 37 wherein said formulation is administered intramuscularly, subcutaneously, ophthalmically, subconjuctivally, intraocularly, via anterior eye chamber injection, intravitreally, intraperitoneally, intrathecally, intracystically, intrapleurally, intranasally, topically, via wound irrigation, intradermally, intrabuccally, intra-abdominally, intra-articularly, intra-aurally, intrabronchially, intracapsularly, intrameningeally, intrapulmonarilly, via inhalation, via endotracheal or endobronchial installation, via direct installation into pulmonary cavities, intraspinally, intrasynovially, intrathoracically, via thoracostomy irrigation, vaginally, epidurally, rectally, intracisternally, intravascularly,intraventricularly, intraosseously, via irrigation of infected bone, and via application as part of any admixture with cement for prosthetic devices.

45. The method of claim 40 wherein said formulation is administered intravenously.

46. The method of claim 40 wherein said formulation is administered intramuscularly, subcutaneously, ophthalmically, subconjuctivally, intraocularly, via anterior eye chamber injection, intravitreally, intraperitoneally, intrathecally, intracystically, intrapleurally, intranasally, topically, via wound irrigation, intradermally, intrabuccally, intra-abdominally, intra-articularly, intra-aurally, intrabronchially, intracapsularly, intrameningeally, intrapulmonarilly, via inhalation, via endotracheal or endobronchial installation, via direct installation into pulmonary cavities, intraspinally, intrasynovially, intrathoracically, via thoracostomy irrigation, vaginally, epidurally, rectally, intracisternally, intravascularly, intraventricularly, intraosseously, via irrigation of infected bone, or via application as part of any admixture with cement for prosthetic devices.

47. The formulation of claim 20, further comprising a second active ingredient selected from one or more of the group consisting of: antifungals; amphotericin B; deoxycholate amphotericin B; flucytosine; terbinafine; antibacterials; antivirals; steroids; nonsteroidal anti-inflammatory drugs (“NSAIDs”); chemotherapeutics; and anti-emitics.

48. The formulation of any of claims 1, 21, 22, 23, 28 or 29 further comprising a second active ingredient selected from one or more of the group consisting of: antifungals; amphotericin B; deoxycholate amphotericin B; flucytosine; terbinafine; antibacterials; antivirals; steroids; nonsteroidal anti-inflammatory drugs (“NSAIDs”); chemotherapeutics; and anti-emitics.

49. The method of claim 37 further comprising administering a second active ingredient selected from one or more of the group consisting of: antifungals; amphotericin B; deoxycholate amphotericin B; flucytosine; terbinafine; antibacterials; antivirals; steroids; nonsteroidal anti-inflammatory drugs (“NSAIDs”); chemotherapeutics; and anti-emitics.

50. The method of claim 40 further comprising administering a second active ingredient selected from one or more of the group consisting of: antifungals; amphotericin B; deoxycholate amphotericin B; flucytosine; terbinafine; antibacterials; antivirals; steroids; nonsteroidal anti-inflammatory drugs (“NSAIDs”); chemotherapeutics; and, anti-emitics.

51. The formulation of claim 1, further characterized by providing a mean maximum plasma concentration (Cₘₐₓ) of posaconazole of at least about 467 ng/ml at steady state, and a mean plasma Area Under the Curve over 24 hours (AUC) value of posaconazole of at least about 9840 ng.hr/ml at steady state, when said formulation is infused over about 1 hour to deliver 100 mg of posaconazole, and repeated at an interval of about 24 hours.

52. The formulation of claim 1, further characterized by providing a mean maximum plasma concentration (Cₘₐₓ) of posaconazole of at least about 852 ng/ml at steady state, and a mean plasma Area Under the Curve over 24 hours (AUC) value of posaconazole of at least about 24,600 ng.hr/ml at steady state, when said formulation is infused over about 1 hour to deliver 200 mg of posaconazole, and repeated at an interval of about 24 hours.

53. The formulation of claim 1, further characterized by providing a mean maximum plasma concentration (Cₘₐₓ) of posaconazole of at least about 1480 ng/ml at steady state, and a mean plasma Area Under the Curve over 24 hours (AUC) value of posaconazole of at least about 24,600 ng.hr/ml at steady state, when said formulation is infused over about 1 hour to deliver at least 200 mg of posaconazole, and repeated at an interval of about 24 hours.

54. The formulation of claim 1, further characterized by providing, after administration of a dosage of about 100 mg of said posaconazole, at least one of: a mean plasma half-life in a range of about 14.9 to about 38.4 hours; and a mean plasma steady state volume of distribution of about 200-500 L.

55. The formulation of claim 1, further characterized as providing, after administration of a dosage of about 200 mg of said posaconazole, at least one of: a mean plasma half-life of about 18.7 to about 35.5 hours; and a mean plasma steady state volume of distribution of about 200-500 L.

56. The formulation of claim 1, further characterized as providing, after administration of a dosage of about 400 mg of said posaconazole, at least one of: a mean plasma half-life of about 18.5 to about 51.4 hours; and a mean plasma steady state volume of distribution of about 200-500 L.

57. The formulation of claim 1, further characterized as providing, after administration of a dosage of about 600 mg of said posaconazole, at least one of: a mean plasma half-life of about 27.2 to about 50.6 hours; and a mean plasma steady state volume of distribution of about 200-500 L.

58. The formulation of claim 1, further characterized as providing a mean posaconazole blood concentration profile substantially similar to that of FIG. 1, when said formulation is infused over about 1 hour to deliver 25-600 mg of posaconazole.

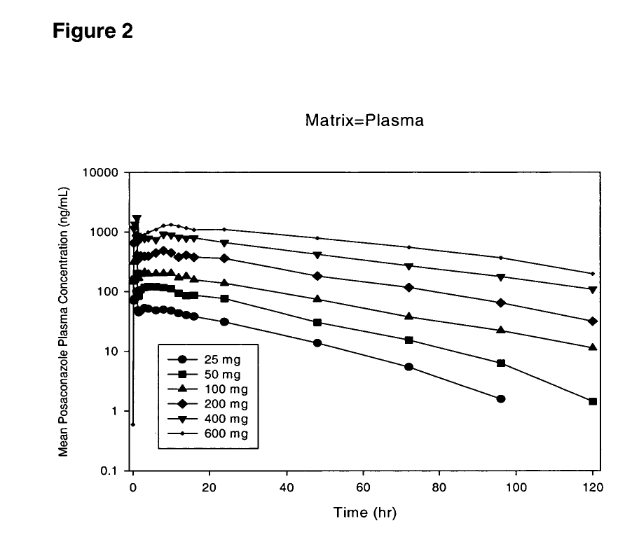

59. The formulation of claim 1, further characterized as providing a mean posaconazole plasma concentration profile substantially similar to that of FIG. 2, when said formulation is infused over about 1 hour to deliver 25-600 mg of posaconazole.

60. The formulation of claim 1, further characterized as providing a ratio of mean posaconazole blood Cₘₐₓ to mean posaconazole plasma Cₘₐₓ of between about 1.5 and about 3.8, when a single dose of said formulation is infused over about 1 hour to deliver 25-600 mg of posaconazole.

61. The formulation of claim 1, further characterized as providing a ratio of mean posaconazole blood Cₘₐₓ to mean posaconazole plasma Cₘₐₓ of between about 2.1 and about 3.3, when a single dose of said formulation is infused over about 1 hour to deliver 25 mg of posaconazole.

62. The formulation of claim 1, further characterized as providing a ratio of mean posaconazole blood Cₘₐₓ to mean posaconazole plasma Cₘₐₓ of between about 1.9 and about 3.8, when a single dose of said formulation is infused over about 1 hour to deliver 50 mg of posaconazole.

63. The formulation of claim 1, further characterized as providing a mean posaconazole blood Cₘₐₓ to mean posaconazole plasma Cₘₐₓ of between about 2.2 and about 3.3, when a single dose of said formulation is infused over about 1 hour to deliver 100 mg of posaconazole.

64. The formulation of claim 1, further characterized as providing a ratio of mean posaconazole blood Cₘₐₓ to mean posaconazole plasma Cₘₐₓ of between about 1.5 and about 3.2, when a single dose of said formulation is infused over about 1 hour to deliver 200 mg of posaconazole.

65. The formulation of claim 1, further characterized as providing a ratio of mean posaconazole blood Cₘₐₓ to mean posaconazole plasma Cₘₐₓ of between about 1.7 and about 3.3, when a single dose of said formulation is infused over about 1 hour to deliver 400 mg of posaconazole.

66. The formulation of claim 1, further characterized as providing a ratio of mean posaconazole blood Cₘₐₓ to mean posaconazole plasma Cₘₐₓ of between about 1.9 and about 3.1, when a single dose of said formulation is infused over about 1 hour to deliver 600 mg of posaconazole.

67. The formulation of claim 1, further characterized as providing a ratio of mean posaconazole blood Cₘₐₓ to mean posaconazole plasma Cₘₐₓ of between about 1.2 and about 2.5, at steady state when said formulation is infused over about 1 hour to deliver 25-600 mg of posaconazole, and repeated on a 24-hour basis.

68. The formulation of claim 1, further characterized as providing a ratio of mean posaconazole blood Cₘₐₓ to mean posaconazole plasma Cₘₐₓ of between about 1.5 and about 2.3, at steady state when said formulation is infused over about 1 hour to deliver 25 mg of posaconazole, and repeated on a 24-hour basis.

69. The formulation of claim 1, further characterized as providing a ratio of mean posaconazole blood Cₘₐₓ to mean posaconazole plasma Cₘₐₓ of between about 1.5 and about 2.4, at steady state when said formulation is infused over about 1 hour to deliver 50 mg of posaconazole, and repeated on a 24-hour basis.

70. The formulation of claim 1, further characterized as providing a ratio of mean posaconazole blood Cₘₐₓ to mean posaconazole plasma Cₘₐₓ of between about 1.7 and about 2.5, at steady state when said formulation is infused over about 1 hour to deliver 100 mg of posaconazole, and repeated on a 24-hour basis.

71. The formulation of claim 1, further characterized as providing a ratio of mean posaconazole blood Cₘₐₓ to mean posaconazole plasma Cₘₐₓ of between about 1.2 and about 2.0, at steady state when said formulation is infused over about 1 hour to deliver 200 mg of posaconazole, and repeated on a 24-hour basis.

72. The formulation of claim 1, further characterized as providing a ratio of mean posaconazole blood Cₘₐₓ to mean posaconazole plasma Cₘₐₓ of between about 1.2 and about 2.2, at steady state when said formulation is infused over about 1 hour to deliver 400 mg of posaconazole, and repeated on a 24-hour basis.

73. The formulation of claim 1, further characterized as providing a ratio of mean posaconazole blood Cₘₐₓ to mean posaconazole plasma Cₘₐₓ of between about 1.3 and about 1.7, at steady state when said formulation is infused over about 1 hour to deliver 600 mg of posaconazole, and repeated on a 24-hour basis.

74. The method of claim 37, wherein said animal is a human.

75. The method of claim 37, wherein said animal is a non-human.

76. The method of claim 40, wherein said animal is a human.

77. The method of claim 40, wherein said animal is a non-human.

78. The formulation of claim 1, further characterized as being bioequivalent to the formulation of any of claims 1, 20, 21, 22, 23, 28 or 29.

79. The method of claim 37, further comprising administering a bolus loading dose of said formulation and then administering an intravenous maintenance dose of said formulation.

80. A method of treating or preventing an infection in an animal in need thereof which comprises administering to said animal an effective amount of posaconazole to provide a mean maximum plasma concentration (Cₘₐₓ) of posaconazole of at least about 467 ng/ml at steady state, and a mean plasma Area Under the Curve over 24 hours (AUC) value of posaconazole of at least about 9840 ng.hr/ml at steady state, when said formulation is infused over about 1 hour to deliver 100 mg of posaconazole, and repeated at an interval of about 24 hours.
